(19) United States
(12) Patent Application Publication
(10) Pub. No.: US 2024/0366762 A1
(43) Pub. Date: Nov. 7, 2024

(54) CHIMERIC FC-ALPHA RECEPTORS AND USES THEREOF

(71) Applicant: Sanquin IP B.V., Amsterdam (NL)

(72) Inventor: Hanke Lottie MATLUNG, Amsterdam (NL)

(21) Appl. No.: 18/557,697

(22) PCT Filed: Apr. 28, 2022
(86) PCT No.: PCT/NL2022/050225

§ 371 Date: Oct. 27, 2023

(30) Foreign Application Priority Data
Apr. 28, 2021 (EP) 21170864.9

(51) Int. Cl. A61K 39/00; A61K 45/06; A61P 35/00; C07K 14/725; C07K 14/735; C07K 16/28; C07K 16/30; C07K 16/32; C12N 5/0783; C12N 5/0787

(52) U.S. Cl. CPC A61K 39/4631; A61K 39/4613; A61K 39/4614; A61K 39/464404; A61K 39/464406; A61K 39/464471; A61K 45/06; A61P 35/00; C07K 14/7051; C07K 14/70535; C07K 16/2863; C07K 16/3084; C07K 16/32; C12N 5/0642; C12N 5/0646; C07K 2319/03; C12N 2510/00

(57) ABSTRACT

The invention relates to polypeptides and chimeric antigen receptors (CARs) comprising an intracellular domain of a Fc alpha Receptor (FcαR), a transmembrane domain of a FcαR, and a ligand-binding domain, to cells comprising and expressing such polypeptides and CARs and to uses thereof.

### FIELD OF THE INVENTION

The invention relates to the field of chimeric receptor and therapy, including cancer therapy. In particular the invention relates to chimeric Fcα receptors.

### BACKGROUND OF THE INVENTION

Remarkable successes have been obtained in tumor therapy by adoptive transfer of in vitro expanded Tumor Infiltrating Lymphocytes (TIL) or T cells expressing chimeric antigen receptors (CAR). CARs contain an ectodomain (a portion of an antibody) specific for antigens found on tumors, coupled to the signaling domains of for instance CD3ζ, and a costimulatory receptor, such as CD28 or 4-1BB. Other signaling domains, such as an Fc-gamma receptor (FcγR), such as FcγRIIIA (CD16A) and C-type lectin-like receptor NKG2D or dimeric receptor DAP10. Expression of CARs in T cells leads to their activation by tumor antigens. Up to 90% complete remissions have been obtained with CAR T cells in certain hematological malignancies. Much less success has been obtained in the treatment of solid tumors, due to limited T cell trafficking and immune resistance mechanism from the tumor microenvironment.

In optimizing CAR therapy in terms of efficacy and safety, and broadening its application to other malignancies, attempts have been made to use CARs as described above in other leukocytes, including NK cells and myeloid cells. NK cells have received the second most attention in CAR research and several clinical trials have been started based on NK cell CAR therapy, mainly aimed at treatment of hematological malignancies, but also treatment of solid tumors (Sievers et al. 2020). CAR-myeloid cells, such as neutrophils and monocytes, have been described, but no clinical trials have been initiated so far. One possibility is to transduce human hematopoietic stem cells (HSCs), followed by expansion and differentiation to generate CAR-expressing granulocytes, monocytes, and macrophages, e.g. as described by De Olivera et al. (2013) for CD19-specific CARs transduced in CD34+HSCs. Roberts et al. describe transduction of HSCs with anti-CD4-CD3ζ CAR and isolation of CAR-expressing neutrophils (1998). WO 2020/223550 describes chimeric fusion proteins or CARs that are expressed on myeloid cells, particularly phagocytic cells. It is described that phagocytic myeloid cells such as macrophages can be engineered with the fusion proteins to have enhanced phagocytic activity.

For this purpose, intracellular signalling domains that comprise a phagocytic signalling domain are used, which domain is preferably derived from a receptor other than MegflO, MerTk, FcRa, and Bail. It is further described that myeloid cells can be engineered to promote T cell activation, but no specific chimeric fusion proteins are described for this purpose. In the experimental section chimeric fusion proteins having a CD8 transmembrane and an FcγR intracellular domain with additional cytosolic domain, such as a PI3K recruitment domain or a CD40 cytosolic portion, are prepared and monocytes and macrophage cell lines are provided with the constructs.

There remains a need in the art for new and improved compositions and methods for immunotherapy, in particular of tumors, such as solid tumors.

### SUMMARY OF THE INVENTION

It is an object of the present invention to provide improved chimeric receptors. It is a further object of the invention to provide methods for improving treatment of tumors in general, and solid tumor specifically.

The invention therefore provides a polypeptide comprising:
an intracellular domain of a Fe alpha Receptor (FcαR),
a transmembrane domain of a FcαR, and
a ligand-binding domain.

In a preferred embodiment, the polypeptide is a chimeric receptor, in particular a chimeric FcαR. In one preferred embodiment, the polypeptide is a chimeric antigen receptor (CAR).

In a further aspect, the invention provides a chimeric antigen receptor (CAR) comprising a polypeptide comprising:
an intracellular domain of a Fe alpha Receptor (FcαR),
a transmembrane domain of a FcαR, and
a ligand-binding domain.

The polypeptide or CAR preferably comprises the indicated domains in the following order: 3′—FcαR intracellular domain—FcαR transmembrane domain—ligand-binding domain—5′. In one preferred embodiment polypeptide or CAR preferably comprises the following domains in the following order: 3′—FcαR intracellular domain—FcαR transmembrane domain—spacer—ligand-binding domain—signal peptide—5′.

In a further aspect, the invention provides a nucleic acid molecule comprising a sequence encoding a polypeptide or CAR according to the invention.

In a further aspect, the invention provides a vector comprising the nucleic acid molecule according to the invention.

In a further aspect, the invention provides a cell comprising the polypeptide, CAR, nucleic acid molecule or vector according to the invention. Said cell further preferably is a cell in which a nucleic acid molecule or vector according to the invention has been introduced. Said cell preferably expresses the polypeptide or CAR according to the invention. Said cell is preferably a hematopoietic stem cell, a myeloid cell or myeloid progenitor cell, or an innate lymphoid cell, more preferably a human hematopoietic stem cell, human myeloid cell or myeloid progenitor cell, or human innate lymphoid cell. Said cells are further preferably selected from granulocytes, neutrophils, monocytes, macrophages, NK cells and combinations thereof. In one embodiment, said cells is a primary cell more preferably an autologous cell isolated from the subject to be treated in accordance with the invention. In another embodiment, said cell is a cell of a cell line, such as NK-92 cell, preferably irradiated NK-92 cell.

In a further aspect, the invention provides a cell, preferably a granulocyte, neutrophil, monocyte, macrophage or NK cell, more preferably a neutrophil or NK cell, comprising a chimeric antigen receptor (CAR) comprising a polypeptide comprising:
an intracellular domain of a Fe alpha Receptor (FcαR),
a transmembrane domain of a FcαR, and
a heterologous ligand-binding domain.

In a further aspect, the invention provides a cell, preferably a granulocyte, neutrophil, monocyte, macrophage or NK cell, more preferably a neutrophil or NK cell, provided with a nucleic acid molecule encoding a CAR comprising an intracellular domain of a Fe alpha Receptor (FcαR), a transmembrane domain of a FcαR, and a heterologous ligand-binding domain.

In a further aspect, the invention provides a population of cells comprising a plurality of cells according to the invention.

In a further aspect, the invention provides a pharmaceutical composition comprising a nucleic acid molecule, vector, polypeptide or CAR according to the invention and at least one pharmaceutically acceptable carrier, diluent or excipient.

In a further aspect, the invention provides a polypeptide, CAR, nucleic acid molecule, vector, cell or population of cells of the invention for use in therapy, in particular for use in immunotherapy. Said cells are preferably myeloid cells or myeloid progenitor cell, hematopoietic stem cells or innate lymphoid cells, more preferably human myeloid cells or myeloid progenitor cell, human hematopoietic stem cells or human innate lymphoid cells. Said cells are preferably selected from granulocytes, neutrophils, monocytes, macrophages, NK cells and combinations thereof. In one embodiment said cells are primary cells, more preferably autologous cells isolated from the subject to be treated in accordance with the invention. In another embodiment, said cells are cells of a cell line, such as NK-92 cells, preferably irradiated NK-92 cells.

In a further aspect, the invention provides a method for immunotherapy in a subject in need thereof comprising administering to the subject thereof a therapeutically effective amount of a polypeptide, CAR, nucleic acid molecule, vector, cell or population of cells of the invention.

In a further aspect, the invention provides a use of a polypeptide, CAR, nucleic acid molecule, vector, cell or population of cells of the invention in the preparation of a medicament for immunotherapy.

In a further aspect, the invention provides a polypeptide, CAR, nucleic acid molecule, vector, cell or population of cells of the invention for use in inducing or stimulating an immune response. Said cells are preferably myeloid cells or myeloid progenitor cell, hematopoietic stem cells or innate lymphoid cells, more preferably human myeloid cells or myeloid progenitor cells, hematopoietic stem cells or innate lymphoid cells. Said cells are preferably selected from granulocytes, neutrophils, monocytes, macrophages, NK cells and combinations thereof. Said cells are further preferably primary cells, more preferably autologous cells isolated from the subject to be treated in accordance with the invention. In another embodiment, said cell is a cell from a cell line, such as the NK-92 cell line.

In a further aspect, the invention provides a method for inducing or stimulating an immune response in a subject in need thereof comprising administering to the subject a polypeptide, CAR, nucleic acid molecule, vector, cell or population of cells of the invention.

In a further aspect, the invention provides a use of a polypeptide, CAR, nucleic acid molecule, vector, cell or population of cells of the invention in the preparation of a medicament for inducing or stimulating an immune response.

In a further aspect, the invention provides a polypeptide, CAR, nucleic acid molecule, vector, cell or population of cells of the invention for use in the treatment of cancer in a subject. In one embodiment, the subject, preferably a human, is suffering from cancer. Said cells are preferably myeloid cells or myeloid progenitor cells, hematopoietic stem cells or innate lymphoid cells, more preferably human myeloid cells or myeloid progenitor cells, hematopoietic stem cells or innate lymphoid cells. Said cells are preferably selected from granulocytes, neutrophils, monocytes, macrophages, NK cells and combinations thereof. Said cells are further preferably primary cells, more preferably autologous cells isolated from the subject to be treated in accordance with the invention. In another embodiment, the cell is a cell from a cell line, such as the NK-92 cell line.

In a further aspect, the invention provides a method for the treatment of cancer in a subject in need thereof, comprising administering to the subject a polypeptide, CAR, nucleic acid molecule, vector, cell or population of cells of the invention.

In a further aspect, the invention provides a use of a polypeptide, CAR, nucleic acid molecule, vector, cell or population of cells of the invention in the preparation of a medicament for the treatment of cancer.

In a further aspect, the invention provides a polypeptide, CAR, nucleic acid molecule, vector, cell or population of cells of the invention for use in the treatment or prevention of a pathogenic infection. Said cells are preferably myeloid cells or myeloid progenitor cells, hematopoietic stem cells or innate lymphoid cells, more preferably human myeloid cells or myeloid progenitor cells, hematopoietic stem cells or innate lymphoid cells. Said cells are preferably selected from granulocytes, neutrophils, monocytes, macrophages, NK cells and combinations thereof. Said cells are further preferably primary cells, more preferably autologous cells isolated from the subject to be treated in accordance with the invention. In another embodiment, the cell is a cell from a cell line, such as the NK-92 cell line.

In a further aspect, the invention provides a method for the treatment or prevention of a pathogenic infection in a subject in need thereof comprising administering to the subject a therapeutically effective amount of a polypeptide, CAR, nucleic acid molecule, vector, cell or population of cells of the invention.

In a further aspect, the invention provides a use of a polypeptide, CAR, nucleic acid molecule, vector, cell or population of cells of the invention in the preparation of a medicament for the treatment or prevention of a pathogenic infection.

In a further aspect, the invention provides a method of producing a cell according to the invention or population of cells according to the invention, comprising
introducing a nucleic acid molecule or vector according to the invention in to a cell or cells of a population of cells, and
allowing expression of the polypeptide or CAR according to the invention. Said cells are preferably myeloid cells or myeloid progenitor cells, hematopoietic stem cells or innate lymphoid cells, more preferably human myeloid cells or myeloid progenitor cells, hematopoietic stem cells or innate lymphoid cells. Said cells are preferably selected from granulocytes, neutrophils, monocytes, macrophages, NK cells and combinations thereof. Said cells are further preferably primary cells, more preferably autologous cells isolated from the subject to be treated in accordance with the invention. In another embodiment, the cell is a cell from a cell line, such as the NK-92 cell line.

### DETAILED DESCRIPTION

As used herein, “to comprise” and its conjugations is used in its non-limiting sense to mean that items following the word are included, but items not specifically mentioned are not excluded. In addition the verb “to consist” may be replaced by “to consist essentially of” meaning that a compound or adjunct compound as defined herein may comprise additional component(s) than the ones specifically identified, said additional component(s) not altering the unique characteristic of the invention.

The articles “a” and “an” are used herein to refer to one or to more than one (i.e., to at least one) of the grammatical object of the article. By way of example, “an element” means one element or more than one element.

The word “approximately” or “about” when used in association with a numerical value (approximately 10, about 10) preferably means that the value may be the given value of 10 more or less 1% of the value.

The use of the alternative (e.g., “or”) should be understood to mean either one, both, or any combination thereof of the alternatives.

The term “therapeutically effective amount,” as used herein, refers to an amount of a compound being administered sufficient to relieve one or more of the symptoms of the disease or condition being treated to some extent. This can be a reduction or alleviation of symptoms, reduction or alleviation of causes of the disease or condition or any other desired therapeutic effect.

As used herein, the term “prevention” refers to precluding or delaying the onset of a disease or condition and/or the appearance of clinical symptoms of the disease or condition in a subject that does not yet experience clinical symptoms of the disease.

The term “treatment” refers to inhibiting the disease or disorder, i.e., halting or reducing its development or at least one clinical symptom of the disease or disorder, and/or to relieving symptoms of the disease or condition. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the term “subject” encompasses humans and animals, preferably mammals. Preferably, a subject is a mammal, more preferably a human.

The term “polypeptide” refers to compounds comprising amino acids joined via peptide bonds. A polypeptide encoded by a nucleic acid sequence is not limited to the amino acid sequence encoded by the nucleic acid sequence, but may include post-translational modifications of the polypeptide.

As used herein with respect to the amino acids sequence of a polypeptide, the terms “N-terminal” and “C-terminal” refer to relative positions in the amino acid sequence of the polypeptide toward the N-terminus and the C-terminus, respectively. “N-terminus” and “C-terminus” refer to the extreme amino and carboxyl ends of the polypeptide, respectively. “Immediately N-terminal” and “immediately C-terminal” refers to a position of a first amino acid sequence relative to a second amino acid sequence where the first and second amino acid sequences are covalently bound to provide a contiguous amino acid sequence.

In amino acid sequences as defined herein amino acids are denoted by single-letter symbols. These single-letter symbols, as well as three-letter symbols, are well known to the person skilled in the art and have the following meaning: A (Ala) is alanine, C (Cys) is cysteine, D (Asp) is aspartic acid, E (Glu) is glutamic acid, F (Phe) is phenylalanine, G (Gly) is glycine, H (His) is histidine, I (Ile) is isoleucine, K (Lys) is lysine, L (Leu) is leucine, M (Met) is methionine, N (Asn) is asparagine, P (Pro) is proline, Q (Gln) is glutamine, R (Arg) is arginine, S (Ser) is serine, T (Thr) is threonine, V (Val) is valine, W (Trp) is tryptophan, Y (Tyr) is tyrosine.

As used herein, a nucleic acid molecule or nucleic acid sequence of the invention comprises a chain of nucleotides of any length, preferably DNA and/or RNA. More preferably, a nucleic acid molecule or nucleic acid sequence of the invention comprises double stranded RNA in order to use RNA interference to degrade target RNA, as explained below. In other embodiments a nucleic acid molecule or nucleic acid sequence of the invention comprises other kinds of nucleic acid structures such as for instance a DNA/RNA helix, peptide nucleic acid (PNA), locked nucleic acid (LNA) and/or a ribozyme. The term nucleic acid molecule includes recombinant and synthetic nucleic acid molecules.

The percentage of identity of an amino acid sequence or nucleic acid sequence, or the term “% sequence identity”, is defined herein as the percentage of residues of the full length of an amino acid sequence or nucleic acid sequence that is identical with the residues in a reference amino acid sequence or nucleic acid sequence after aligning the two sequences and introducing gaps, if necessary, to achieve the maximum percent identity. As used herein sequence identity is calculated on the basis of consecutive amino acids of the subject amino acid sequence. Methods and computer programs for the alignment are well known in the art, for example “Align 2”. Programs for determining nucleotide sequence identity are also well known in the art, for example, the BESTFIT, FASTA and GAP programs. These programs are readily utilized with the default parameters recommended by the manufacturer.

The terms “specifically binds” and “specific for” as used herein refer to the interaction between a ligand binding domain and its ligand, including the interaction between an antibody, or antigen-binding part thereof, and its epitope.

The terms means that said ligand binding domain, preferentially binds to said ligand over other ligands. Although the ligand binding domain may non-specifically bind to other portions, amino acid sequences or ligands, the binding affinity of said ligand binding domain for its ligand is significantly higher than the non-specific binding affinity of said ligand binding domain for other portions, amino acid sequences or ligands.

The term “antibody” as used herein, refers to an immunoglobulin protein comprising at least a heavy chain variable region (VH), paired with a light chain variable region (VL), that is specific for a target epitope.

A “antigen binding part” of an antibody is defined herein as a part that shares the property of the said antibody that it is specific for the target epitope. I.e. the antigen binding part is capable of binding the same antigen as said antibody, albeit not necessarily to the same extent. In one embodiment, an antigen binding part of an antibody comprises at least a heavy chain variable domain (VH). Non-limiting examples of antigen binding parts of an antibody are a single domain antibody, a single chain antibody, a nanobody, an unibody, a single chain variable fragment (scFv), a Fab fragment and a F(ab′)₂ fragment.

As used herein the term “nanobody” or “antigen binding nanobody” refers to a single-domain antibody consisting of a single monomeric variable antibody domain which is able to bind selectively to the target antigen.

As used herein, the term “scFv” refers to a molecule comprising an antibody heavy chain variable region (VH) and an antibody light chain variable region (VL), which may be connected by a linker, for instance having the general structure NH₂-V_{L}-linker-V_{H}-COOH or NH₂-V_{H}-linker-V_{L}-COOH.

As used herein, the term “affimer” refers to a single-chain-based antibody analogue capable of specifically binding to the antigen, and which is based on a cystatin cysteine protease inhibitor scaffold (see Johnson et al. 2012, incorporated herein by reference).

As used herein the term “spacer” refers to an amino acid sequence that connects two domains in a polypeptide or CAR and may provide for proper expression and functionality of the polypeptide or CAR.

As used herein the term “domain” refers to an amino acids sequence that has a certain function in the polypeptide or CAR of the invention. Examples of such domains include, but are not limited to, alia, the FcαR intracellular domain, the FcαR transmembrane domain, the ligand-binding domain, a spacer between transmembrane domain and ligand-binding domain, a signal peptide, a tag and a dimerization domain.

As used herein the terms “chimeric receptor” and “chimeric antigen receptor” or “CAR” refer to a recombinant polyprotein comprising at least an extracellular ligand binding domain that binds specifically to a ligand, such as an antigen or a target, a transmembrane domain and at least one intracellular signalling domain.

These at least three domains are derived from at least two different naturally occurring polypeptides or proteins. The chimeric antigen receptor or CAR may comprises other sequences, such as a signal peptide sequence, one or more spacer domains and a costimulatory domain.

The present inventors have developed a tuneable CAR construct comprising a transmembrane and intracellular part of the Fcα-receptor (FcαR) and a variable extracellular domain. Activation of the FcαR on neutrophils by binding of IgA-isotype tumor antigen-specific antibodies leads to a strong anti-tumor effect, which is in general stronger compared to Fey-receptor signalling induced by IgG, the isoform of therapeutic antibodies now broadly applied clinically. By making use of the intracellular domain of this activating Fcα-receptor it is hypothesized that the anti-tumor effector functions of the CAR are amplified as compared to Fey-receptor signalling using a Fey-receptor based CAR.

It is further hypothesized that one of the effector functions of the neutrophils expressing a CAR having a FcαR intracellular domain is trogocytosis, a recently described effector mechanism of neutrophils against antibody-opsonized tumor cells. Trogocytosis is a necrotic, lytic cell death that is characterized by fragmentation of target cells, initiated by membrane uptake of membrane fragments of the target cells by the neutrophils.

The CAR of the present invention comprising an FcαR intracellular and transmembrane domain can be tuned for recognizing a wide variety of antigens, including antigens of hematopoietic malignancies and solid tumor antigens, resulting in high therapeutic potential for targeting cancers of different origins. Hence, the CAR is modular in its extracellular tumor recognizing domain for optimal flexibility. Furthermore, the CAR technology is designed to be applicable for use in multiple innate effector cells, including neutrophils, monocyte/macrophages and NK cells.

Innate cells, including neutrophils and macrophages, are essential effectors of the immune system. Harnessing these innate cells against tumor cells is of longstanding interest, especially in the case of solid tumors, where existing T cell therapies have limited success. CARs, receptors initially designed to specifically redirect T cell activity towards tumor antigens, have been used successfully in targeting haematological cancers, but less so in solid tumors. The present CAR technology is believed to be especially suitable and broadly applicable in cases where T cell-based therapies are limited. Specifically directing innate effector functions against the tumor by the expression of target specific CARs opens up new possibilities for long-lasting tumor control. In particular, tumor-associated macrophages (TAMs) and polymorphonuclear cells (PMNs) are known to infiltrate solid tumors well and CAR-TAMs/PMNs are believed to polarize the tumor microenvironment (TME) into anti-tumor vs pro-tumor. In addition, CAR-TAMs/PMNs have the potential to generate an adaptive response on top of the direct anti-tumor effect by cross-presentation of tumor antigens or co-stimulation of T cells. Although the use of innate CARs may provide an obstacle for maintaining a durable response, resulting from their rapid turnover, this may actually add to restriction of serious side effects as seen for CAR T cells.

The examples herein describe the development of a CAR targeted against GD2, expressed on neuroblastoma cells. The CAR construct is designed as a GD2-recognizing domain, consisting of the single chain fragment variable (scFv) part derived from the commercially available anti-GD2 antibody (clone 14.18) and a transmembrane and intracellular part of the Fcα-receptor (FcαR). As shown in the examples herein, maturation inducible cells can be successfully transduced to express the CAR (more than 80% positive cells) and differentiated into neutrophil like cells. Moreover, neutrophil-like cells expressing the GD2-CAR are able to specifically kill GD2+ neuroblastoma cells. In particular the cells were shown to induce cytotoxicity of GD2-positive neuroblastoma cells, without the need for anti-GD2 opsonization. It has further been shown that the FcαR intracellular domain is necessary for the function of the GD2-CAR. Importantly, the present inventors have found that CAR constructs with an FcαR intracellular and transmembrane domain in neutrophil like cells show far better effectivity than CAR constructs with an FcγRIIA intracellular and transmembrane domain: GD2-FcαR CAR neutrophil like cells were shown to be capable of effectively killing GD2+ target cells in all target to effector cell ratio's tested, whereas GD2-FcγR CAR in these neutrophil like cells did not induce killing (see FIG. 11B). Strikingly, the combination of the GD2-FcαR CAR and GD2-FcγR CAR also resulted in enhanced cytotoxicity as compared to GD2-FcαR CAR alone, which demonstrates a synergistic effect of the combination.

In addition, CAR constructs with a Her2/neu or EGFR recognizing domain have been prepared, and the neutrophil like cells provided with these CAR constructs were shown to be effective against EGFR+ and Her2/neu+ epidermoid carcinoma and breast cancer cell lines A431 and SKBR3 respectively, without the need for anti-EGFR and anti-Her2/neu opsonization. The cytotoxic capacity of the Her2/neu-FcαR CAR for non-opsonized cells was surprisingly found to be higher than that of wildtype neutrophil like cells for trastuzumab-opsonized target cells.

Finally, the present inventors have further successfully expressed the GD2-CAR in NK cells.

In a first aspect, the invention therefore provides a polypeptide comprising:
an intracellular domain of a Fe alpha Receptor (FcαR),
a transmembrane domain of a FcαR, and
a ligand-binding domain.

In a preferred embodiment, the polypeptide is a chimeric receptor, in particular a chimeric FcαR. In one preferred embodiment, the polypeptide is a chimeric antigen receptor (CAR).

Also provided is a chimeric antigen receptor (CAR) comprising a polypeptide comprising:
an intracellular domain of a Fe alpha Receptor (FcαR),
a transmembrane domain of a FcαR, and
a ligand-binding domain.

In preferred embodiments, the CAR consists of the polypeptide, i.e. the CAR comprises an intracellular domain of a Fe alpha Receptor (FcαR), a transmembrane domain of a FcαR, and a ligand-binding domain.

The polypeptide and CAR of the present invention comprise an intracellular and transmembrane domain of a Fe alpha Receptor (FcαR). The intracellular and transmembrane domain of a polypeptide or CAR according to the invention are preferably of a human FcαR. FcαR is also referred to as immunoglobulin alpha Fc receptor and human FcαR exists in multiple isoforms, including isoform A.1, isoform A.2, isoform A.3, isoform B, isoform B-delta-S2, isoform U02, isoform L10, isoform U09, isoform U10, isoform U11 and isoform U13. These FcαR isoforms and their sequences are known in the art. In the examples herein, an intracellular and transmembrane domain of the canonical isoform of isoform A.1 are used. The sequence of FcαR isoform A.1 is depicted in FIG. 1. However, the sequences of any FcαR can be used in a polypeptide or CAR of the invention. In a preferred embodiment, the human FcαR is human FcαR isoform A.1, isoform A.2, isoform A.3, isoform U02, isoform L10, isoform U10, isoform U11 or isoform U13.

An “intracellular domain of a FcαR” as used herein refers to an intracellular domain that is capable of initiating FcαR signaling. The polypeptide or CAR according to the present invention is thus capable of FcαR signaling. FcαR signaling is initiated when the ligand-binding domain is bound a ligand. Hence, “capable of FcαR signaling” means that FcαR signaling is initiated when the ligand-binding domain of the polypeptide or CAR is bound a ligand. The FcαR intracellular domain is well known to a person skilled in the art. It comprises the 41 C-terminal amino acids of human FcαR. These are amino acids 247-287 of FcαR isoform A.1, i.e. the sequence ENWHSHTALNKEASADVAEPSWSQQMCQPGLTFARTPSVCK, or the corresponding amino acids in other FcαR isoforms. Hence, the intracellular domain of a FcαR as present in a polypeptide or CAR of the invention preferably comprises a sequence of amino acids 247-287 of the FcαR isoform A.1 sequence as shown in FIG. 1, or the corresponding sequence of a FcαR isoform other than isoform A.1, or a sequence that is at least 90% identical to said sequence. Said FcαR isoform other than isoform A.1 is preferably isoform A.2, isoform A.3, isoform U02, isoform L10, isoform U10, isoform U11 or isoform U13. The intracellular domain as present in a polypeptide or CAR of the invention is preferably capable of initiating FcαR signaling. Said sequence is preferably at least 95% identical to amino acids 247-287 of said sequence of FcαR isoform A.1 as shown in FIG. 1, more preferably at least 97%, more preferably at least 98%, more preferably at least 99%. In a particularly preferred embodiment, the intracellular domain of a FcαR comprises amino acids 247-287 of said sequence of FcαR isoform A.1 as shown in FIG. 1, more preferably it consists of amino acids 247-287 of said sequence of FcαR isoform A.1 as shown in FIG. 1.

In preferred embodiments, the CAR does not comprise a further intracellular domain in addition to the intracellular domain of a FcαR. In particular, the CAR does not comprise a further functional intracellular domain in addition to the intracellular domain of a FcαR. Hence, no further functional domains are present intracellularly when the CAR is expressed by a cell, preferably a granulocyte, neutrophil, monocyte, macrophage or NK cell, more preferably a neutrophil or NK cell. Non-functional sequence such as a linking sequence or spacer may be present.

In particularly preferred embodiments, the intracellular and transmembrane portions of the CAR consists of an intracellular domain and transmembrane domain of a FcαR, optionally separated by a linking sequence or spacer. In further preferred embodiments, the intracellular and transmembrane portions of the CAR consists of an intracellular domain and transmembrane domain of a FcαR. The “intracellular and transmembrane portions of the CAR” as used herein refers to the portions of the CAR that are intracellular and transmembrane when the CAR is expressed by a cell, preferably a granulocyte, neutrophil, monocyte, macrophage or NK cell, more preferably a neutrophil or NK cell.

A “transmembrane domain of a FcαR” as used herein refers to a transmembrane domain of FcαR. The FcαR transmembrane domain is well known to a person skilled in the art. It comprises the 19 amino acids immediately N-terminal to the FcαR intracellular domain. These are amino acids 228-246 of FcαR isoform A.1, i.e. the sequence LIRMAVAGLVLVALLAILV, or the corresponding amino acids in other FcαR isoforms. Hence, the transmembrane domain of a FcαR as present in a polypeptide or CAR of the invention preferably comprises a sequence of amino acids 228-246 of the FcαR isoform A.1 sequence as shown in FIG. 1, or the corresponding sequence of a FcαR isoform other than isoform A.1, or a sequence that is at least 90% identical to said sequence. Said FcαR isoform other than isoform A.1 is preferably isoform A.2, isoform A.3, isoform U02, isoform L10, isoform U10, isoform U11 or isoform U13. Said sequence is preferably at least 95% identical to amino acids 228-246 of said sequence of FcαR isoform A.1 as shown in FIG. 1, more preferably at least 97%, more preferably at least 98%, more preferably at least 99%. In a particularly preferred embodiment, the transmembrane domain of a FcαR comprises amino acids 228-246 of said sequence of FcαR isoform A.1 as shown in FIG. 1, more preferably it consists of amino acids 228-246 of said sequence of FcαR isoform A.1 as shown in FIG. 1.

Hence, a preferred polypeptide or CAR of the invention comprises a sequence of amino acids 228-287 of the FcαR isoform A.1 sequence as shown in FIG. 1, or the corresponding sequence of a FcαR isoform other than isoform A.1, or a sequence that is at least 90% identical to said sequence. Said FcαR isoform other than isoform A.1 is preferably isoform A.2, isoform A.3, isoform U02, isoform L10, isoform U10, isoform U11 or isoform U13. Said sequence is preferably at least 95% identical to amino acids 228-287 of said sequence of FcαR isoform A.1 as shown in FIG. 1, more preferably at least 97%, more preferably at least 98%, more preferably at least 99%. In a particularly preferred embodiment, the transmembrane domain of a FcαR comprises amino acids 228-287 of said sequence of FcαR isoform A.1 as shown in FIG. 1, more preferably it consists of amino acids 228-287 of said sequence of FcαR isoform A.1 as shown in FIG. 1.

The polypeptide or CAR of the invention may comprise further partial sequences of a FcαR, such as part of the extracellular domain of a FcαR. For instance, the polypeptide or CAR of the invention optionally comprises between 1 and 50 contiguous amino acids of the extracellular domain of a FcαR, preferably 1 to 50 contiguous amino acids immediately N-terminal to the transmembrane domain of FcαR. This corresponds to amino acids 178-227 of the FcαR isoform A.1 sequence as shown in FIG. 1, but may also be the 178-227 of an FcαR isoform other than A.1. In one embodiment, the polypeptide or CAR of the invention comprises between 5 and 30 amino acids immediately N-terminal to the transmembrane domain of FcαR, i.e. at least 5 contiguous amino acids of amino acids 198-227 immediately N-terminal of the transmembrane domain of FcαR isoform A.1 as shown in FIG. 1, or the corresponding amino acids of a FcαR isoform other than A.1. Such as 5, 6, 7, 8, 9, 10, 11, 12, 13, 14, 15, 16, 17, 18, 19 or 20 contiguous amino acids immediately N-terminal of the transmembrane domain of FcαR isoform A.1 as shown in FIG. 1, or the corresponding amino acids of a FcαR isoform other than A.1. Such additional amino acids immediately N-terminal of the transmembrane domain may provide for flexibility of the polypeptide or CAR of the invention.

The term “ligand-binding domain” as used herein refers to a domain that binds specifically to a ligand. The ligand-binding domain is an heterologous ligand binding domain which, as used herein, means that it is a ligand-binding domain other than the extracellular domain of a FcαR. The ligand-binding domain can be any domain that can be bound by a ligand of choice. In particular, the ligand-binding domain can be the binding partner of any cell surface antigen or any soluble ligand. The versatility in the ligand-binding domain allows to select an appropriate ligand for any specific application. This way, activation of FcαR signaling by the polypeptide or CAR of the invention can be initiated at a selected time, a selected location/cell type, or both. Preferred examples of suitable extracellular ligand-binding domains are a ligand binding domain specific for a soluble ligand, a ligand binding domain specific for a cell surface antigen and a combination thereof. Preferred examples of cell surface antigens are tumor antigens, myeloid derived suppressor cell antigen and pathogenic antigens. As used herein “tumor antigen” refers to any antigen expressed on cells of a tumor. A tumor antigen is also referred to as a tumor-associated antigen (TAA). As used herein “myeloid derived suppressor cell antigen” refers to any antigen expressed on MDSCs. As used herein “antigen expressed on myeloid derived suppressor cell” refers to immune cells from the myeloid lineage that expand in pathological situations such as cancer and chronic infections and that possess immunosuppressive properties. As such, MDSCs have infection or tumor-promoting activity, depending on the environment. In one preferred embodiment the MDSC is a tumor-associated MDSC, such as an MDSC present in the tumor microenvironment. As used herein “pathogenic antigen” refers to any antigen expressed by a microorganism or parasite, which includes, but are not limited to, viruses, bacteria, parasites, fungi and parasites. In a preferred embodiment, the ligand-binding domain is specific for a tumor-antigen or a myeloid derived suppressor cell (MDSC) antigen. The following types of cell surface antigens can be the target of a ligand-binding domain of a polypeptide or CAR of the invention: tumor or MDSC specific antigens; antigens that have a higher level of expression on tumor cells or MDSCs as compared to the expression level on non-tumor cells and non-MDSCs; antigens that are expressed on both tumor cells or MDSCs and non-tumor cells and non-MDSCs, but whereby activation of cells expressing the polypeptide or CAR of the invention induced by non-tumor cells and non-MDSCs results in side-effects that are acceptable; antigens that are expressed on both tumor cells or MDSCs and non-tumor cells or non-MDSCs, but that are specific for tumor cells or MDSCs in combination with one or more other antigens; and antigens expressed on cells surrounding a tumor.

In one embodiment, the ligand-binding domain comprises a moiety selected from the group consisting of:
an antibody or antigen binding part of an antibody, such as a single chain variable fragment (scFv), nanobody or affimer specific for a cell surface antigen;
an extracellular Fc-binding domain of an Fc receptor or a ligand-binding fragment thereof,
a ligand-binding domain specific for a soluble ligand,
an epitope for an antibody that can crosslink the polypeptide or CAR without involvement of a surface antigen or molecule;
a domain recognizing a moiety, such as biotin, FITC or a peptide epitope, which moiety can be coupled to a molecule (e.g. antibody or scFv) specific for a cell surface antigen, such as a tumor antigen,
a moiety, such as biotin, that can be crosslinked by an agent with multiple binding sites for that moiety, such as streptavidin (resulting in clustering of multiple polypeptides or CARs upon addition of said agent).

In one preferred embodiment, a cell surface antigen is a tumor antigen.

In one preferred embodiment, the ligand-binding domain is an antibody or antigen binding part of an antibody, including a scFv and a nanobody or an affimer specific for said tumor antigen. For instance, any known therapeutic antibody specific for a tumor antigen, or an antigen binding part thereof, such as scFv, can be used as the ligand-binding domain of a polypeptide or CAR of the invention.

Preferred examples of tumor antigens are GD2, EGFR, HER2/Neu, TAG-72, calcium-activated chloride channel 2, including TMEM16A, 9D7, Ep-CAM, EphA3, mesothelin, SAP-1, BAGE family, MC1R, prostate-specific antigen, CML66, TGF-βRII, MUC1, CD5, CD19, CD20, CD30, CD33, CD47, CD52, CD152 (CTLA-4), CD274 (PD-L1), CD273 (PD-L2), CD340 (ErbB-2), TPBG, CA-125, MUC1, and immature laminin receptor.

In one preferred embodiment, the tumor antigen is selected from GD2, EGFR and HER2/Neu. GD2 is a disialoganglioside expressed on tumors of neuroectodermal origin, including human neuroblastoma (about 91-100% of patients) and melanoma (about 2.4-14% of patients). GD2 is further expressed on small cell lung cancer (SCLC; about >50% of patients), Ewing sarcoma (about 40-90% of patients, osteosarcoma (about 88% of patients), glioma (about 80% of patients), retinoblastoma (about 40% of patients) and breast cancer, bladder cancer (expression depending on stage and subtype). Epidermal growth factor receptor (EGFR) is a transmembrane protein. Mutations that lead to EGFR overexpression have been associated with a number of cancers, including colorectal carcinoma, head and neck cancer (about 80-100% of patients), non-small cell lung cancer (NSCLC; about 40% of patients), in particular metastatic NSCLC, anal cancers and glioblastoma (about 50% of patients). HER2/Neu is an oncogene that is expressed by certain types of breast cancer.

A skilled person is well capable of identifying soluble ligands and their binding partners that can be used as ligand-binding domain in a polypeptide or CAR according to the invention. As soluble ligand for instance soluble forms of ligands can be used that bind to the ligand-binding domain of the polypeptide or CAR of the invention and as such provide a stop signal to the polypeptide- or CAR-expressing cell.

In one embodiment, the ligand-binding domain comprises an extracellular Fc-binding domain of an Fc receptor or a ligand-binding fragment thereof. Instead of a cell-surface antigen as ligand binding domain the polypeptide or CAR comprises extracellularly the antibody-recognizing domain of an Fc-receptor, i.e. CD16. This way the polypeptide or CAR is designed to recognize therapeutic antibodies directed against tumor cells or MDSCs but at the same time benefit from amplified signalling induced by the transmembrane and intracellular FcαR domain of the polypeptide or CAR.

A combination of a ligand binding-domain specific for a soluble ligand comprised in the polypeptide or CAR of the invention and a ligand binding domain comprised in a separate molecule (e.g. antibody or scFv) specific for a cell surface antigen, such as a tumor antigen, is also possible. In that case FcαR signalling will only be induced if both the soluble ligand and the cell surface antigen are present. For instance, a ligand binding domain of a polypeptide or CAR of the invention can comprise a domain recognizing e.g. a peptide neo-epitope or to a Biotin or FITC moiety. Use will then be made of another antibody (a “switch” antibody) directed to a (tumor) surface antigen on a tumor, which contains the peptide neo-epitope, Biotin or FITC moiety. As a consequence, activation of FcαR signalling will only occur if, in addition to the cell surface antigen targeted by the switch antibody, the switch antibody itself is also present. Examples of such technologies that can be used with the polypeptide or CAR of the present invention are described in Ma et al. (2016) and Mitwasi et al. (2020), which are incorporated herein by reference, and permits temporary control of the receptor (turning it on and off only when desired) as well as quantitative control (by in- or decreasing the concentration of the switch antibody).

A polypeptide or CAR according to the invention may comprise further sequences or domains in addition to the intracellular domain of a FcαR, transmembrane domain of a FcαR, and ligand-binding domain. Examples of such sequences and domains are one or more linking sequences or spacer between the FcαR intracellular domain and the FcαR transmembrane domain and/or between the FcαR transmembrane domain and the ligand-binding domain, a signal peptide sequence, one or more tags and a dimerization motif.

In one preferred embodiment, a spacer is located between the FcαR transmembrane domain and the ligand-binding domain. Such spacer preferably comprises up to 300 amino acids, such as 10-300 amino acids. In one preferred embodiment, the spacer consists of at least 200 amino acids. In principle such spacer can be any random amino acid sequence. Examples of suitable spacer sequence are IgG1, IgG2 or IgG4 based spacers, e.g. based on the CH₂CH₃ hinge region optionally comprising one or more mutations to reduce binding to Fcγ receptors, and extracellular domains lacking FcγR binding activity, such as CD28 and CD8a domains. Suitable examples are described in Guedan et al. (2019), which is incorporated by reference herein. Another example of a suitable spacer is a Siglec-4 based spacer (as described in Schafer et al. (2020), which is incorporated by reference herein). In one preferred embodiment, the spacer comprises or is the CH₂CH₃ hinge region of human IgG, preferably IgG1, IgG2 or IgG4, more preferably IgG1, optionally containing one or more mutations. This CH₂CH₃ hinge region of human IgG and its sequence are well known in the art, and is e.g. amino acids 98-329 of the sequence of GenBank accession no. AAC82527.1.

In one preferred embodiment, the polypeptide or CAR of the invention comprises a signal peptide. A signal peptide that is able to direct polypeptide to the cell membrane may be present to stimulate a cell to translocate the polypeptide or CAR to the cell membrane. Signal peptides are well known in the art and a skilled person is well capable of selecting a suitable signal peptide. Such signal peptide preferably comprises up to 50 amino acids. In one preferred embodiment, the signal peptide consists of 10-40 amino acids or 15-35 amino acids. In one embodiment the signal peptide is the human immunoglobulin heavy chain signal peptide having the sequence MEFGLSWLFLVAILKGVQCE (amino acids 1-19 of the sequence of GenBank accession no. AAA587335.1).

In another embodiment, the polypeptide or CAR of the invention comprises a tag, preferably comprised in the extracellular domain, such as a small peptide epitope. Such tag can be used to eliminate cells expressing the polypeptide or CAR of the invention, by specifically targeting the tag, e.g. CAR T cells specific for the tag. Suitable tags can be identified by a person skilled in the art and one suitable tag and procedure to eliminate CAR expressing cells, that can be used in a polypeptide or CAR of the present invention, is described in Koristka et al. (2019), which is incorporated by reference herein. As another example, such tag can be used to attached the ligand binding domain comprising a moiety recognizing the tag to the remainder of the polypeptide or CAR.

In preferred embodiments, the CAR does not comprise a further intracellular domain in addition to the intracellular domain of a FcαR. In particular, the CAR does not comprise a further functional intracellular domain in addition to the intracellular domain of a FcαR. Hence, no further functional domains are present intracellularly when the CAR is expressed by a cell, preferably a granulocyte, neutrophil, monocyte, macrophage or NK cell, more preferably a neutrophil or NK cell. Non-functional sequence such as a linking sequence or spacer may be present.

In particularly preferred embodiments, the intracellular and transmembrane portions of the CAR consists of an intracellular domain and transmembrane domain of a FcαR, optionally separated by a linking sequence spacer.

Further provided is a nucleic acid molecule comprising a sequence encoding a polypeptide or CAR according to the invention. Also provided is a vector comprising a nucleic acid molecule according to the invention. In a preferred embodiment, the vector is a viral vector, e.g., a lentiviral vector or a retroviral vector. In another preferred embodiment, the vector comprises or is a transposon. Said nucleic acid molecule or vector may additionally comprise other components, such as means for high expression levels such as strong promoters, for example of viral origin, that direct expression in the specific cell in which the vector is introduced, and signal sequences. In a preferred embodiment, the nucleic acid molecule or vector comprises one or more of the following components: a promoter that drives expression in innate cells, such as the SFFV promoter, a C-terminal signal peptide such as from the GMCSF protein or the CD8 protein for targeting to the plasma membrane and a polyadenylation signal.

Further provided is the polypeptide or CAR of the invention when expressed by a cell. Also provided is a cell, preferably an isolated cell, comprising the nucleic acid molecule or vector according to the invention. Also provided is a population of cells according to the invention. Said population of cells preferably comprises a plurality of cell according to the invention. The cell is preferably a human cell. Cells in a population of cells are preferably human cells.

The cell is preferably a hematopoietic stem cell, a maturation inducible cell, a human pluripotent stem cell (hPSC), an induced pluripotent stem cell (iPSC), a myeloid cell or myeloid progenitor cell, or an innate lymphoid cell. Innate lymphoid cells (ILCs) are innate counterparts of T cells that contribute to immune responses by secreting effector cytokines and regulating the functions of other innate and adaptive immune cells and include NK cells, ILC1s, ILC2s, ILC3s and lymphoid tissue inducer (LTi) cells. More preferably, the cell is a hematopoietic stem cell, a hPSC, an iPSC, a granulocyte, including a neutrophil, basophil, and eosinophil, a monocyte, macrophage, myeloblast, erythrocyte, dendritic cell or mast cell, or an NK cell, or a combination thereof. The cells in a population of cells are preferably myeloid cells or myeloid progenitor cell, human hematopoietic stem cells, hPSC, human iPSC, and/or an innate lymphoid cell, more preferably selected from hematopoietic stem cells, granulocytes, including neutrophils, basophils, and eosinophils, monocytes, macrophages, myeloblasts, erythrocytes, dendritic cells and mast cell, NK cells, and a combination thereof. In one preferred embodiment, the cell is a granulocyte, neutrophil, monocyte, macrophage or NK cell, or a combination thereof. In preferred embodiments, the cell is a neutrophil. In other preferred embodiments, the cell is a NK cell. In one preferred embodiment, the cells in a population of cells are selected from granulocytes, neutrophils, monocytes, macrophages, NK cells, or a combination thereof. In preferred embodiments, the cells in a population of cells are neutrophils. In other preferred embodiments, the cells in a population of cells are NK cells. The cell, in particular neutrophil of NK cell, can be of any origin, including a cell of a cell line, a primary cell or a cell differentiated from any maturation inducible cell, hPSC or iPSC. As used herein a “primary cell” refers to a cell that is obtained directly from an organism, preferably a human. The primary cells can be cultured to undergo proliferation or expansion or the cells are directly modified and used as described herein below. In one embodiment, the primary cells are directly modified. The primary cell, preferably hematopoietic stem cell, myeloid cell or myeloid progenitor cell, or innate lymphoid cell, more preferably hematopoietic stem cell, granulocyte, neutrophil, monocyte, macrophage or NK cell, or a combination thereof, is for instance isolated from blood or healthy tissue of the subject. Isolation of cell from blood or healthy tissue can be performed using standard methods in the art for isolation of such cells. A preferred example of a cell line is an NK cell line, more preferably the NK-92 cell line. This cell line is suitability for CAR therapy and is described in Tang et al. (2018), which is incorporated by reference herein. If the cell is a hematopoietic stem cell, myeloid progenitor cell, maturation inducible cell, hPSC or iPSC, the cell can be, and is preferably, differentiated or expanded and differentiated to generate CAR-expressing myeloid cells, in particular granulocytes, neutrophils, monocytes, and/or macrophages or NK cells.

The cell, preferably hematopoietic stem cell, maturation inducible cell, hPSC, iPSC, myeloid cell or myeloid progenitor cell, or innate lymphoid cell, is preferably an engineered cell or recombinantly modified cell. As used herein the term “engineered cell” and “recombinantly modified cell” refer to a cell into which a foreign, i.e., non-naturally occurring, nucleic acid has been introduced, in particular to a cell wherein a nucleic acid molecule or vector of the invention has been introduced. I.e. the cell is preferably a cell in which the nucleic acid molecule or the vector according to the invention has been introduced. A nucleic acid molecule or vector may be introduced into the cell, preferably immune cells, by any method known in the art, such as by transfection, transduction, e.g. lentiviral transduction or retroviral transduction, DNA electroporation, or RNA electroporation. The nucleic acid molecule or vector is either transiently, or, stably provided to the cell. Methods for transfection, transduction or electroporation of cells with a nucleic acid are known to the skilled person.

The cell of the invention preferably comprises the polypeptide or CAR of the invention, more preferably expresses the polypeptide or CAR of the invention. Also provided is therefore a cell comprising a chimeric antigen receptor (CAR) comprising a polypeptide comprising an intracellular domain of a Fe alpha Receptor (FcαR), a transmembrane domain of a FcαR, and a heterologous ligand-binding domain.

The polypeptide or CAR of the invention is preferably expressed at the cell surface. I.e. the cell is genetically modified to express the polypeptide or CAR of the invention. The intracellular domain of a FcαR in the polypeptide or CAR of the invention is preferably intracellular when the polypeptide or CAR is expressed by a cell. The transmembrane domain of a FcαR in the polypeptide or CAR of the invention is preferably transmembrane when the polypeptide or CAR is expressed by a cell. The ligand-binding domain is preferably extracellular when the polypeptide or CAR is expressed by a cell.

In one embodiment, the cell is an autologous cell isolated from a patient, in particular a patient that is to be treated in accordance with the present invention.

In one embodiment, the population of cells is a population of autologous cells isolated from a patient, in particular a patient that is to be treated in accordance with the present invention. In one embodiment, said patient is a patient suffering from cancer, in particular suffering from a cancer that is treated in accordance with the present invention. In another embodiment, the cell is a cell from a cell line, such as the NK-92 cell line.

Also provided is a pharmaceutical composition comprising a nucleic acid molecule, vector, polypeptide or CAR according to the invention and at least one pharmaceutically acceptable carrier, diluent or excipient. Also provided is a pharmaceutical composition comprising a cell or population of cells according to the invention and at least one pharmaceutically acceptable carrier, diluent and/or excipient. By “pharmaceutically acceptable” it is meant that the carrier, diluent or excipient must be compatible with the other ingredients of the formulation and not deleterious, e.g. toxic, to the recipient thereof. In general, any pharmaceutically suitable additive which does not interfere with the function of the active compounds can be used. A pharmaceutical composition according to the invention is preferably suitable for human use.

Examples of suitable carriers comprise a solution, lactose, starch, cellulose derivatives and the like, or mixtures thereof. In a preferred embodiment said suitable carrier is a solution, for example saline. The pharmaceutical composition is preferably a formulation for parenteral administration, in particular a transfusion formulation. Formulations for parenteral administration include intraarticular, intramuscular, intravenous, intraventricular, intraarterial, intrathecal and subcutaneous administration. Further, the pharmaceutical composition may be administered to a subject in hospital via infusion or via injection from a healthcare professional. Compositions for parenteral administration may for example be solutions of the nucleic acid molecule, vector, polypeptide, CAR, cell or population of cells of the invention in sterile isotonic aqueous buffer. Where necessary, the parenteral formulations may include for instance solubilizing agents, stabilizing agents and/or a local anesthetic to ease the pain at the site of the injection.

The polypeptides, CARs, nucleic acid molecules, vectors, cells and populations of cells of the invention are advantageously used in therapy, preferably immunotherapy. Provided is therefore a polypeptide, CAR, nucleic acid molecule, vector, cell or population of cells of the invention for use in therapy and for use in immunotherapy. Also provided is a method for immunotherapy in a subject in need thereof comprising administering to the subject thereof a therapeutically effective amount of a polypeptide, CAR, nucleic acid molecule, vector, cell or population of cells of the invention. In one embodiment, said immunotherapy is tumor immunotherapy. Also provided is a polypeptide, CAR, nucleic acid molecule, vector, cell or population of cells of the invention for use in inducing or stimulating an immune response, in particular in the treatment of cancer. Also provided is a method for inducing or stimulating an immune response in a subject in need thereof, in particular in the treatment of cancer, comprising administering to the subject a polypeptide, CAR, nucleic acid molecule, vector, cell or population of cells of the invention. In a preferred embodiment, any such method comprises administration of a cell or population of cells according to the invention. In a preferred embodiment said immunotherapy, in particular tumor immunotherapy comprises adoptive cell transfer, more preferably adoptive myeloid cell transfer or adoptive innate lymphoid cell transfer, wherein myeloid cell or innate lymphoid cell is a cell as defined herein above. Preferably said myeloid cells and/or or innate lymphoid cells are selected from granulocytes, neutrophils, monocytes, macrophages, NK cells or combinations thereof.

As used herein “adoptive cell transfer” refers to the transfer of cells of the invention into a patient, in particular a patient suffering from cancer. In particular, “adoptive myeloid cell transfer” refers to the transfer of myeloid cells of the invention into a patient and “adoptive innate lymphoid cell transfer” refers to the transfer of innate lymphoid cells of the invention into a patient. The cells may have originated from the patient itself or may have come from another individual.

The cells have been engineered or recombinantly modified in accordance with the present invention to express a polypeptide or CAR of the invention. Adoptive myeloid cell transfer preferably comprises transfer of autologous myeloid cells derived from the subject or patient to be treated. Adoptive innate lymphoid cell transfer” preferably comprises transfer of autologous innate lymphoid cells, in particular NK cells, derived from the subject or patient to be treated, or cells of the NK-92 cell line.

As used herein “immunotherapy” refers to treatment of an individual suffering from a disease or disorder by inducing or enhancing an immune response in said individual. Tumor immunotherapy relates to inducing or enhancing an individual's immune response against a tumor and/or cells of said tumor.

Immunotherapy according to the invention can be either for treatment or prevention, preferably it is for treatment, in particular of cancer or a tumor.

Also provided is a polypeptide, CAR, nucleic acid molecule, vector, cell or population of cells of the invention for use in the treatment of cancer in a subject. In one embodiment, the subject, preferably a human, is suffering from cancer. Also provided is a method for the treatment of cancer in a subject in need thereof, comprising administering to the subject a polypeptide, CAR, nucleic acid molecule, vector, cell or population of cells of the invention. Said treatment of cancer preferably comprise administering an effective amount of cells of the invention, i.e. cells that express the polypeptide or CAR of the invention, to the subject, preferably human suffering from cancer. Said cells are preferably myeloid cells, or innate lymphoid cells, more preferably cells selected from granulocytes, neutrophils, monocytes, macrophages, NK cells or combinations thereof. Said cells are further preferably autologous cells isolated from the subject, wherein subsequently a nucleic acid molecule or vector of the invention has been introduced, thereby providing autologous cells that express the polypeptide or CAR of the invention. In another embodiment, the cell is a cell from a cell line, such as the NK-92 cell line.

Cancer that can be treated using therapy based on a polypeptide or CAR according to the invention and/or a cell, preferably myeloid cell, or innate lymphoid cells, more preferably autologous or cell line myeloid cells or innate lymphoid cells, such as granulocytes, neutrophils, monocytes, macrophages, NK cells or combinations thereof, wherein a nucleic acid molecule or vector according to the invention has been introduced or expressing a polypeptide or CAR according to the invention can be any type of tumor, including solid tumors, haematological tumors, primary tumors, secondary tumors, advanced tumors and metastases. Non-limiting examples tumors that can be treated or prevented in accordance with the invention are acute myeloid leukemia (AML), chronic myeloid leukemia (CML), chronic lymphocytic leukemia (CLL), acute lymphoblastic leukemia (ALL), chronic myelomonocytic leukemia (CMML), lymphoma, multiple myeloma, eosinophilic leukemia, hairy cell leukemia, Hodgkin lymphoma, non-Hodgkin lymphoma, large cell immunoblastic lymphoma, plasmacytoma, lung tumors, small cell lung cancer, non-small cell lung cancer, pancreatic tumors, breast tumors, liver tumors, brain tumors, skin tumors, bone tumors, including Ewing sarcoma and osteosarcoma, colon tumors, rectal tumors, anal tumors, tumors of the small intestine, stomach tumors, gliomas, endocrine system tumors, adrenal gland tumors, including neuroblastoma, thyroid tumors, esophageal tumors, gastric tumors, uterine tumors, urinary tract tumors and urinary bladder tumors, kidney tumors, renal cell carcinoma, prostate tumors, gall bladder tumors, tumors of the head or neck, ovarian tumors, cervical tumors, tumor of the eye, including retinoblastoma, glioblastoma, melanoma, chondrosarcoma, fibrosarcoma, endometrial, esophageal, eye or gastrointestinal stromal tumors, liposarcoma, nasopharyngeal, thyroid, vaginal and vulvar tumors.

In one embodiment, the tumor is a solid tumor. It has been found by the present inventors that the CAR technology of the present invention is especially suitable for treatment of solid tumors. In particular, myeloid cells such as tumor-associated macrophages (TAMs) and polymorphonuclear cells (PMNs) are known to infiltrate solid tumors well and CAR-TAMs/PMNs are believed to polarize the tumor microenvironment (TME) into anti-tumor vs pro-tumor. In addition to that, FcαR signalling is initiated when the ligand-binding domain is bound, and consequently innate effector functions. Hence, in one preferred embodiment, the cancer is selected from the group consisting of neuroblastoma, melanoma, small cell lung cancer (SCLC), Ewing sarcoma, osteosarcoma, glioma, retinoblastoma, breast cancer, bladder cancer, colon cancer, head and neck cancer, non-small cell lung cancer (NSCLC), anal cancers, and glioblastoma.

In one preferred embodiment, the cancer that is treated in accordance with the invention is neuroblastoma. Approximately 25-35 children with a median age of 3 years are diagnosed with neuroblastoma in the Netherlands each year, and about 800 children in the US. About 50% of these diagnosed children fall into the high risk group and undergo intensive multimodal treatment. Recently, antibody-based anti-GD2 immunotherapy was integrated in treatment protocols for patients with neuroblastoma, improving the overall survival for in particular high risk neuroblastoma patients. As a mechanism of action, anti-GD2-opsonized tumor cells are killed through antibody-dependent cellular cytotoxicity (ADCC), a process mediated by various FcγR-expressing immune cells, including the innate effector cells neutrophils, macrophages and NK cells. Although the survival rates have improved significantly by the addition of anti-GD2 antibodies to the treatment regime, still around 50% of patients will relapse and succumb to the disease.

Especially these high risk neuroblastoma patients are therefore in need of more potent and targeted approaches, which initially were introduced in the form of adoptive transfer of chimeric antigen receptor (CAR) T cells. Although showing promise in early phase clinical trials, CAR T cell activity against neuroblastoma has not been as robust as is the case for their use in haematological malignancies.

Especially suboptimal T cell persistence, potency, and an immunosuppressive tumor microenvironment were considered serious challenges in the use of CAR T cells against neuroblastoma. As neutrophils, myeloid and NK cells are considered important effector cells driving the success of anti-GD2 treatment they are particularly useful for treatment of neuroblastoma in accordance with the present invention.

In another preferred embodiment, the cancer that is treated in accordance with the invention is melanoma.

In another preferred embodiment, the cancer that is treated in accordance with the invention is ewing sarcoma.

In another preferred embodiment, the cancer that is treated in accordance with the invention is breast cancer, in particular Her2/neu+ and/or EGFR+ breast cancer.

The polypeptides, CARs, nucleic acid molecules, vectors and in particular cells expressing a polypeptide or CAR of the invention, are advantageously combined with one or more further therapeutic agents or treatments. Such further therapy or therapeutic agent can be any anti-cancer or immune modulatory agent or treatment known in the art. In one embodiment, the CAR, nucleic acid molecule, vector or cell is combined with a therapeutic antibody, checkpoint inhibitor, cytokine, chemotherapeutic agent, or T cell based therapy. In a preferred embodiment, the combination is with an immunotherapeutic agent or immunotherapy, in particular a therapeutic antibody, checkpoint inhibitor, cytokine, or T cell based therapy.

Preferred, but non-limiting, examples of checkpoints are cytotoxic T-lymphocyte antigen-4 (CTLA-4), programmed death-1 (PD-1), PD-ligand 1 (PD-L1), PD-L2, Signal-regulatory protein alpha (SIRPa), CD47, T-cell immunoglobulin- and mucin domain-3-containing molecule 3 (TIM3), lymphocyte-activation gene 3 (LAG3), killer cell immunoglobulin-like receptor (KIR), CD276, CD272, A2AR, VISTA and indoleamine 2,3 dioxygenase (IDO). A therapeutic antibody or checkpoint inhibitor that is combined with a polypeptide, CAR or cell expressing a polypeptide or CAR according to the invention is preferably selected from the group consisting of an anti-CTLA4 antibody, an anti-PD-1 antibody, an anti-PD-L1 antibody, an anti-PD-L2 antibody, an anti-SIRPa antibody, an anti-CD47 antibody, an anti-TIM3 antibody, an anti-LAG3 antibody, an anti-CD276 antibody, an anti-CD272 antibody, an anti-KIR antibody, an anti-A2AR antibody, an anti-VISTA antibody, anti TIGIT antibody an anti-IDO antibody.

Further therapeutic antibodies that are advantageously combined with a polypeptide, CAR or cells expressing a polypeptide or CAR according to the invention are an anti-GD2 antibody, an anti-EGFR antibody, an anti-Her2/Neu antibody, anti-CD20 antibody, an anti-CD3 antibody, anti-TNFalpha antibody, an anti-CD147 antibody, an anti-IL8 antibody, an anti-MUC18 antibody, an anti-MUC1, an anti-alpha-4-beta-1 (VLA-4) and alpha-4-beta-7 antibody, an anti-VLA-1 integrin antibody, an anti-lymphotoxin beta receptor (LTBR) antibody, an anti-TGF-. beta antibody, an anti-IL-12 p40 antibody, an anti-VEGF antibody, an anti-HER receptor family antibody, an anti-CD11a antibody, an anti-IL15 antibody, an anti-CD40L antibody, an anti-CD80 antibody, an anti-CD23 antibody, an anti-macrophage migration factor (MIF) antibody, anti-VE cadherin antibodies, an anti-CD22 antibody, an anti-CD30 antibody, an anti-IL15 antibody, anti-intercellular adhesion molecule-1 (ICAM-1) (CD54) antibody, an anti-fibroblast growth factor receptor 3 (FGFR-3) antibody, an anti-gamma interferon antibody, anti-IL-12 antibody, an anti-Ep-CAM antibody. Suitable antibodies used for combination with a treatment of the present invention are dinutuximab, nivolumab, pembrolizumab, lambrolizumab, ipilimumab, lirilumab, trastuzumab, cetuximab, pertuzumab, panitumumab, necitumumab, bevacizumab, ramucirumab, olaratumab, atezolizumab, ado-trastuzumab emtansine, denosumab, avelumab, cemiplimab, durvalumab, enfortumab vedotin, trastuzumab deruxtecan, sacituzumab govitecan.

In some preferred embodiments, the therapeutic antibody is an antibody that is specific for the ligand or antigen that is recognized by the heterologous ligand-binding domain of the CAR. In other preferred embodiments, the therapeutic antibody is an antibody that is specific for another ligand or antigen than the ligand or antigen that is recognized by the heterologous ligand-binding domain of the CAR. Preferably both ligands and/or antigens are expressed by the specific cells, in particular cancer or tumor cells, that are targeted by the CAR. For instance, as demonstrated in the Examples, an anti-EGFR antibody is advantageously combined with a Her2/Neu-CAR.

A used herein, the term “chemotherapeutic agent” refers to a cytotoxic agent which is used for chemotherapy of cancer. Non-limiting examples of a chemotherapeutic agent that is advantageously combined with a polypeptide, CAR or cells expressing a polypeptide or CAR according to the invention include an alkylating agent, e.g. cyclophosphamide, mechlorethamine, chlorambucil, melphalan, dacarbazine, nitrosoureas and temozolomide, an anthracycline, e.g. doxorubicin, daunorubicin, epirubicin, idarubicin, mitoxantrone and valrubicin, a cytoskeletal disruptor, e.g. docetaxel, paclitaxel, abraxane and taxotere, a topoisomerase 1 inhibitor, e.g. irinotecan and topotecan, topoisomerase H inhibitor, e.g. etoposide, teniposide and tafluposide, a kinase inhibitor, e.g. bortezomib, erlotinib, gefitinib, imatinib, vemurafenib and vismodegib, a nucleotide analogue, e.g. azacitidine, azathioprine, capecitabine, cytarabine, doxifluridine, fluorouracil, gemcitabine, hydroxyurea, mercaptopurine, methotrexate and tioguanine, a platinum-based agent, e.g. cisplatin, carboplatin and oxaliplatin, and a vinca alkaloid derivative, e.g. vinblastine and vincristine.

Non-limiting examples of cytokines that are advantageously combined with a polypeptide, CAR or cells expressing a polypeptide or CAR according to the invention include granulocyte-macrophage colony stimulating factor (GM-CSF), interleukin (IL)-2, IL-3, IL-4, and IL-12, an interferon, such as IFN-α, IFN-β, IFN-ε, IFN-γ, IFN-κ, IFN-λ, IFN-τ and IFN-ω, tumor necrosis factor alpha (TNFα), G-CSF.

As demonstrated in the Examples (see FIG. 5) neutrophil like cells expressing GD2-, EGFR- or Her2/neu-FcαR CAR according to the invention surprisingly show increased cytotoxicity against GD2+, EGFR+ and Her2/neu+ target cells, respectively, if the treatment is combined with antibodies that target the respective tumor antigen or another antigen that is expressed by the target cells. Hence, in preferred embodiments, in particular in immunotherapy, including treatment of cancer, the cell or population of cells of the invention are combined with an anti-tumor antigen antibody. In some preferred embodiments, such anti-tumor antigen antibody is an antibody that is specific for the ligand or antigen, in particular a tumor antigen, that is recognized by the heterologous ligand-binding domain of the CAR. In other preferred embodiments, the therapeutic antibody is an antibody that is specific for another ligand or antigen than the ligand or antigen that is recognized by the heterologous ligand-binding domain of the CAR. Preferably both ligands and/or antigens are expressed by the specific cells, in particular cancer or tumor cells, that are targeted by the CAR. For instance, as demonstrated in the Examples, an anti-EGFR antibody is advantageously combined with a Her2/Neu-CAR.

The polypeptides, CARs, nucleic acid molecules, vectors and in particular cells expressing a polypeptide or CAR of the invention, are also advantageously used in the treatment of pathogenic infections. As used herein “pathogenic antigen” refers to any antigen expressed by a microorganism or parasite, which includes, but are not limited to, viruses, bacteria, parasites, fungi and parasites. Examples of pathogenic bacteria that that can be treated in accordance with the invention include, but are not limited to, Listeria, Escherichia, Chlamydia, Rickettsia, Mycobacterium, Staphylococcus, Streptococcus, Pneumococcus, Meningococcus, Klebsiella, Pseudomonas, Legionella, Diphtheria, Salmonella, Vibrio, Clostridium, Bacillus, Yersinia, and Leptospira bacteria. Examples of pathogenic viruses that that can be treated in accordance with the invention include, but are not limited to, A, B or C hepatitis, herpes virus (for instance VZV, HSV-I, HAV-6, HSV-II, CMV, EpsteinBarr-virus), adenovirus, influenza virus, flaviviruses, echovirus, rhinovirus, coxsackie virus, coronavirus, respiratory syncytial virus (RSV), rotavirus, Morbillivirus, rubella virus, parvovirus, vaccinia virus, HTLV virus, dengue virus, papillomavirus, poliovirus, rabies virus and human immunodeficiency virus (HIV virus; e. g., type I and II). Examples of pathogenic fungi that that can be treated in accordance with the invention include, but are not limited to, Candida (e.g., albicans, krusei, glabrata, tropicalis), Aspergillus (e.g., fumigatus, niger), Cryptococcus neoformans, Histoplasma capsulatum, Mucorales, Blastomyces dermatitidis, Paracoccidioides brasiliensis, and Coccidioides immitis. Examples of pathogenic parasites that can be treated in accordance with the invention include, but are not limited to, Entamoeba histolytica, Plasmodium (e.g. falciparum, vivax), Entamoeba, Giardia, Balantidium coli, Acanthamoeba, Cryptosporidium, Pneumocystis carinii, Babesia microti, Trypanosoma (e.g. brucei, cruzi), Leishmania (e.g. donovani), and Toxoplasma gondii.

In one embodiment the nucleic acid molecule, vector or cells are comprised in or used in combination with a vaccine. Provided is therefore a vaccine comprising a nucleic acid molecule, vector or cells according to the invention for use in a method for inducing or stimulating an immune response in a subject in need thereof. The method comprises administering the vaccine to the subject. Also provided is therefore a vaccine comprising a nucleic acid molecule, vector or cells according to the invention for use in a method for the treatment or prevention of a pathogenic infection in a subject in need thereof. The method comprises administering the vaccine to the subject. A vaccine according to the invention preferably comprises further constituents, such as pharmaceutically acceptable carriers or excipients and one or more adjuvants.

In some embodiments nucleic acid molecule, vector or cells are comprised in or used in combination with another CAR comprising a non-FcαR intracellular and transmembrane domain, preferably a CAR with an FcγR intracellular and/or transmembrane domain, more preferably both an FcγR intracellular and transmembrane domain. The extracellular ligand-binding domain of such FcγR-CAR and the heterologous ligand-binding domain of the FcαR-CAR according to the invention may be the same or different. In some preferred embodiments, the FcγR-CAR comprises an extracellular ligand-binding domain that recognizes the same ligand or antigen that is recognized by the heterologous ligand-binding domain of the FcαR-CAR according to the invention. In further preferred embodiments, the extracellular ligand-binding domain that recognizes the same ligand or antigen that is recognized by the heterologous ligand-binding domain of the FcαR-CAR according to the invention comprise or consist of the same ligand-binding domain.

In other preferred embodiments, the therapeutic antibody is an antibody that is specific for another ligand or antigen than the ligand or antigen that is recognized by the heterologous ligand-binding domain of the CAR. Preferably both ligands and/or antigens are expressed by the specific cells, in particular cancer or tumor cells, that are targeted by the CAR. For instance, as demonstrated in the Examples, an anti-EGFR antibody is advantageously combined with a Her2/Neu-CAR.

Also provided is a method of producing a cell according to the invention or population of cells according to the invention, comprising
introducing a nucleic acid molecule or vector according to the invention in to a cell or cells of a population of cells, and
allowing expression of the polypeptide or CAR according to the invention.

Said cells are preferably myeloid cells or myeloid progenitor cells, hematopoietic stem cells or innate lymphoid cells, more preferably human myeloid cells or myeloid progenitor cell, hematopoietic stem cells or innate lymphoid cells. Said cells are preferably selected from granulocytes, neutrophils, monocytes, macrophages, NK cells and combinations thereof. Said cells are further preferably primary cells, more preferably autologous cells isolated from the subject to be treated in accordance with the invention. In another embodiment, the cell is a cell from a cell line, such as the NK-92 cell line.

Features may be described herein as part of the same or separate aspects or embodiments of the present invention for the purpose of clarity and a concise description. It will be appreciated by the skilled person that the scope of the invention may include embodiments having combinations of all or some of the features described herein as part of the same or separate embodiments.

The invention will be explained in more detail in the following, non-limiting examples.

### BRIEF DESCRIPTION OF THE DRAWINGS

FIG. 1: Amino acids sequence of Fcα receptor (uniprot P24071-1).

FIG. 2: A. GD2-CAR design. B. Sequence of exemplary CAR.

FIG. 3: Expression of GD2-FcαR-CAR in NB4 cells. NB4 cells were differentiated towards neutrophil-like cells by 7 days stimulation with All-Trans Retinoic Acid (indicated as “atra”). Expression of the GD2-FcαR-CAR was quantified by flow cytometry; n=4 (A), and furthermore analysed by western blot (B). Cells were stained with the use of primary antibody BiotinSP AffiniPure f(ab)2 (Jackson Immunoresearch) and secondary antibody Streptavidin Alexafluor 647 (Thermo Fischer Scientific). The red arrow indicates the CAR at the expected molecular weight. The green fragments at the bottom are a loading control.

FIG. 4: Cytotoxicity of GD2-positive neuroblastoma cell lines LAN-1, NMB and IMR-32 by parental and FcαR-CAR-expressing NB4 cells. NB4 cells were differentiated towards neutrophil-like cells by 7 days stimulation with All-Trans Retinoic Acid. Tumor cells were harvested and labelled with 51chromium, after which NB4 cells and tumor cells were incubated together in a target:effector ratio of 1:50 for 4 hours at 37 degrees. Tumor cells were either left untreated [−], or opsonized with the anti-GD2 antibody dinutuximab [Dimab]. Cytotoxicity was determined compared to spontaneous and maximum release of 51chromium from the tumor cells and calculated as [(experimental value−spontaneous release)/(maximum release− spontaneous value)*100%]. Significance was tested by Anova; p<0.05 was considered statistical significant. *=p<0.05; **=p<0.01; ns=not significant.

FIG. 5: Increased cytotoxicity of FcαR CAR NB4 cells combined with tumor antigen targeting antibody towards GD2+, EGFR+ and Her2/neu+ target cells. NB4 cells were differentiated towards neutrophil-like cells by 7 days stimulation with All-Trans Retinoic Acid. Tumor cells NMB, LAN-1, IMR-32, TC-71, A431 and A431 Her2/neu were harvested and labelled with ⁵¹chromium, after which NB4 cells and tumor cells were incubated together in several target:effector ratios, namely 1:25, 1:50, 1:100 and 1:200, for 4 hours at 37 degrees. Tumor cells were either left untreated [−], or opsonized with the anti-GD2 antibody dinutuximab [Dimab] or anti-EGFR antibody cetuximab [Cmab]. Cytotoxicity was compared to spontaneous and maximum release of 51chromium from the tumor cells and calculated as [(experimental value−spontaneous release)/(maximum release−spontaneous value)*100%]. Significance was tested by Anova; p<0.05 was considered statistical significant. *=p<0.05; **=p<0.01; ns=not significant.

FIG. 6: Differentiation of NB4 cells, ROS production and adhesion. NB4 cells were differentiated towards neutrophil-like cells by 7 days stimulation with All-Trans Retinoic Acid. The level of differentiation was analysed by staining for several surface markers by flow cytometry; left panel percentage of positive cells, right panel MFI, n=4 (A). ROS production upon use of various stimuli was quantified by the amount of hydrogen peroxide released by the NB4 cells as measured by Amplex Red assay on a plate reader (B). The ability of the NB4 cells to adhere to a plate, as a measure for CD18 function, was measured by the amount of calcein labeled cells adhering to a 96-wells plate upon several stimuli and was detected on a plate reader; n=3. Lysis of the NB4 cells was added as a 100% control to control for labeling efficiency of the NB4 cells by calcein; n=3 (C).

FIG. 7: GD2-CAR is antigen dependent. NB4 cells were differentiated towards neutrophil-like cells by 7 days stimulation with All-Trans Retinoic Acid. Expression of GD2 and GD2 KO in LAN-1 target cells was quantified by flow cytometry; n=1 (A). (B) Tumor cells (LAN-1 WT and GD2 KO) were harvested and labelled with 51chromium, after which GD2-FcαR CAR NB4 cells and tumor cells were incubated together in several target:effector ratios, namely 1:25, 1:50, 1:100 and 1:200, for 4 hours at 37 degrees. Cytotoxicity was compared to spontaneous and maximum release of ⁵¹chromium from the tumor cells and calculated as [(experimental value−spontaneous release)/(maximum release−spontaneous value)*100%]. N=4 independent experiments. (C) Tumor cell trogocytosis by NB4 cells was tested by quantification of membrane uptake by flow cytometry. LAN WT or GD2 KO cells were stained with the membrane dye DiD and incubated with WT NB4 cells or GD2-FcαR CAR NB4 cells for 90 minutes at 37 degrees. After co-incubation the cells were fixed and measured on Canto flow cytometer. N=3 independent experiments. Significance was tested by Anova; p<0.05 was considered statistical significant. *=p<0.05; **=p<0.01; ns=not significant.

FIG. 8: Functionality of GD2-FcαR CAR is depending on expression of the FcαR cytoplasmic tail. NB4 cells were differentiated towards neutrophil-like cells by 7 days stimulation with All-Trans Retinoic Acid. Expression of GD2-FcαR (red bars) and GD2-FcαR Δcyt. CAR (blue bars) in NB4 cells was quantified by flow cytometry. Cells were therefor stained with the primary antibody BiotinSP AffiniPure F(ab′)2 (Jackson Immunoresearch) and Streptavidin alexafluor 647 (Life technologies). N=2 independent experiments (A). (B) Tumor cells (LAN-1 WT) were harvested and labelled with ⁵¹chromium, after which GD2-FcαR (red bars) and GD2-FcαR Δcyt. CAR (blue bars) NB4 cells and tumor cells were incubated together in several target:effector ratios, namely 1:25, 1:50, 1:100 and 1:200, for 4 hours at 37 degrees. Cytotoxicity was compared to spontaneous and maximum release of ⁵¹chromium from the tumor cells and calculated as [(experimental value−spontaneous release)/(maximum release−spontaneous value)*100%]. N=8 independent experiments. Significance was tested by Anova; p<0.05 was considered statistical significant. *=p<0.05; **=p<0.01; ns=not significant.

FIG. 9: FcαR CAR is effective against EGFR tumor antigen. NB4 cells were differentiated towards neutrophil-like cells by 7 days stimulation with All-Trans Retinoic Acid. (A) Expression of EGFR-FcαR in NB4 cells was quantified by flow cytometry. Cells were therefor stained with the primary antibody BiotinSP AffiniPure F(ab′)2 (Jackson Immunoresearch) and Streptavidin alexafluor 647 (Life technologies). WT NB4 were stained as controls. Histograms of staining are shown on the left; quantification of 2 independent experiments is shown on the right. (B) Tumor cells (A431) were harvested and labelled with ⁵¹chromium, after which WT NB4 cells (grey bars) and EGFR-FcαR (red bars) NB4 cells and tumor cells were incubated together in several target:effector ratios, namely 1:25, 1:50, 1:100 and 1:200, for 4 hours at 37 degrees. Target cells were either left unopsonized, or opsonized with anti-EGFR antibody cetuximab (Cmab, 1 μg/ml). Cytotoxicity was compared to spontaneous and maximum release of ⁵¹chromium from the tumor cells and calculated as [(experimental value−spontaneous release)/(maximum release−spontaneous value)*100%]. N=4 independent experiments. (C) Tumor cell trogocytosis by NB4 cells was tested by quantification of membrane uptake by flow cytometry. A431 cells were stained with the membrane dye DiD and incubated with WT NB4 cells (grey bars) or EGFR-FcαR CAR NB4 cells (red bar) for 90 minutes at 37 degrees. Target cells were either left unopsonized, or opsonized with anti-EGFR antibody cetuximab (1 μg/ml) After co-incubation the cells were fixed and measured on Canto flow cytometer. N=9 independent experiments. Significance was tested by Anova; p<0.05 was considered statistical significant. * p<0.05; **=p<0.01; ns=not significant.

FIG. 10: FcαR CAR is effective against Her2/neu tumor antigen. NB4 cells were differentiated towards neutrophil-like cells by 7 days stimulation with All-Trans Retinoic Acid. (A) Expression of Her2/neu-FcαR in NB4 cells was quantified by flow cytometry. Cells were therefor stained with the primary antibody BiotinSP AffiniPure F(ab′)2 (Jackson Immunoresearch) and Streptavidin alexafluor 647 (Life technologies). WT NB4 were stained as controls. Histograms of staining are shown on the left; quantification of 2 independent experiments is shown on the right. (B) Tumor cells (SKBR3) were harvested and labelled with ⁵¹chromium, after which WT NB4 cells (grey bars) and Her2/neu-FcαR (red bars) NB4 cells and tumor cells were incubated together in several target:effector ratios, namely 1:25, 1:50, 1:100 and 1:200, for 4 hours at 37 degrees. Target cells were either left unopsonized, or opsonized with anti-Her2/neu antibody trastuzumab (Tmab, 1 μg/ml). Cytotoxicity was compared to spontaneous and maximum release of ⁵¹chromium from the tumor cells and calculated as [(experimental value−spontaneous release)/(maximum release−spontaneous value)*100%]. N=6 independent experiments. (C) Tumor cell trogocytosis by NB4 cells was tested by quantification of membrane uptake by flow cytometry. SKBR3 cells were stained with the membrane dye DiD and incubated with WT NB4 cells (grey bars) or Her2/neu-FcαR CAR NB4 cells (red bar) for 90 minutes at 37 degrees. Target cells were either left unopsonized, or opsonized with anti-Her2/neu antibody trastuzumab (1 μg/ml) After co-incubation the cells were fixed and measured on Canto flow cytometer. N=10 independent experiments. Significance was tested by Anova; p<0.05 was considered statistical significant. * p<0.05; **=p<0.01; ns=not significant.

FIG. 11: GD2-FcαR CAR outperforms GD2-FcγR CAR. NB4 cells were differentiated towards neutrophil-like cells by 7 days stimulation with All-Trans Retinoic Acid. (A) Expression of GD2-FcαR CAR and GD2-FcγRIIA CAR in NB4 cells was quantified by flow cytometry. Cells were therefor stained with the primary antibody BiotinSP AffiniPure F(ab′)2 (Jackson Immunoresearch) and Streptavidin alexafluor 647 (Life technologies). WT NB4 were stained as controls (B) Tumor cells (LAN-1) were harvested and labelled with ⁵¹chromium, after which GD2-FcαR NB4 cells (red bars) and GD2-FcγR CAR (grey bars) NB4 cells and tumor cells were incubated together in several target:effector ratios, namely 1:25, 1:50, 1:100 and 1:200, for 4 hours at 37 degrees. Cytotoxicity was compared to spontaneous and maximum release of ⁵¹chromium from the tumor cells and calculated as [(experimental value−spontaneous release)/(maximum release−spontaneous value)*100%]. N=4 independent experiments. Significance was tested by Anova; p<0.05 was considered statistical significant. *=p<0.05; ** p<0.01; ns=not significant.

FIG. 12: Dual expression of GD2-FcαR CAR and GD2-FcγR CAR enhances cytotoxicity against GD2+ tumor. NB4 cells were differentiated towards neutrophil-like cells by 7 days stimulation with All-Trans Retinoic Acid. (A) Expression of dual CAR constructs in NB4 cells, analyzed by flow cytometry. N=2 independent experiments. (B) Cytotoxicity assay: tumor cells were harvested and labelled with ⁵¹chromium, after which NB4 cells and tumor cells were incubated together in several target:effector ratios, namely 1:25, 1:50, 1:100 and 1:200, for 4 hours at 37 degrees. Tumor cells were either left untreated [−], or opsonized with the anti-GD2 antibody dinutuximab [Dimab]. Cytotoxicity was compared to spontaneous and maximum release of ⁵¹chromium from the tumor cells and calculated as [(experimental value−spontaneous release)/(maximum release− spontaneous value)*100%]. N=6 independent experiments. Significance was tested by Anova; p<0.05 was considered statistical significant. *=p<0.05; **=p<0.01; ns=not significant.

FIG. 13: FcαR CAR leads to additional 62-integrin activation compared to FcγR activation by mAb. NB4 cells were differentiated towards neutrophil-like cells by 7 days stimulation with All-Trans Retinoic Acid. Cytotoxicity assay: tumor cells were harvested and labelled with ⁵¹chromium, after which WT NB4 cells (A) or GD2-FcαR CAR NB4 cells (B) and tumor cells were incubated together in several target:effector ratios, namely 1:25, 1:50, 1:100 and 1:200, for 4 hours at 37 degrees. Tumor cells were either left untreated [−], or opsonized with the anti-GD2 antibody dinutuximab [Dimab]. CD11b was blocked by saturating amounts anti-CD11b (44A, 10 μg/ml); CD18 was blocked by saturating amounts of anti-CD18 antibody (1B4, 10 μg/ml). Cytotoxicity was determined compared to spontaneous and maximum release of ⁵¹chromium from the tumor cells and calculated as [(experimental value−spontaneous release)/(maximum release−spontaneous value)*100%]. N=3 independent experiments. Significance was tested by Anova; p<0.05 was considered statistical significant. *=p<0.05; **=p<0.01; ns=not significant.

FIG. 14: GD2-FcαR CAR is expressed in NK-92 cells. NK-92 cells were lentivirally transduced to express GD2-FcαR CAR. Expression was analyzed by flow cytometry. Cells were therefor stained with the primary antibody BiotinSP AffniniPure F(ab′)2 (Jackson Immunoresearch) and Streptavidin alexafluor 647 (Life technologies).

### EXAMPLES

### Materials and Methods

### Cells, Culture, and Antibodies

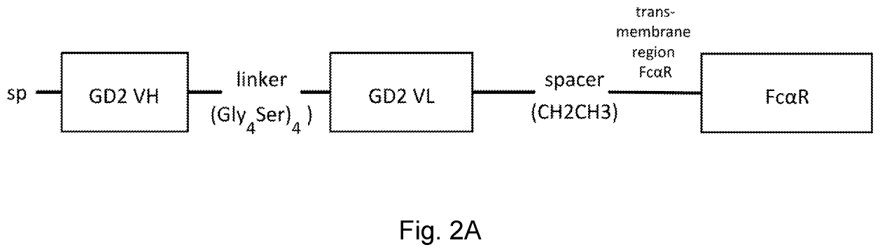

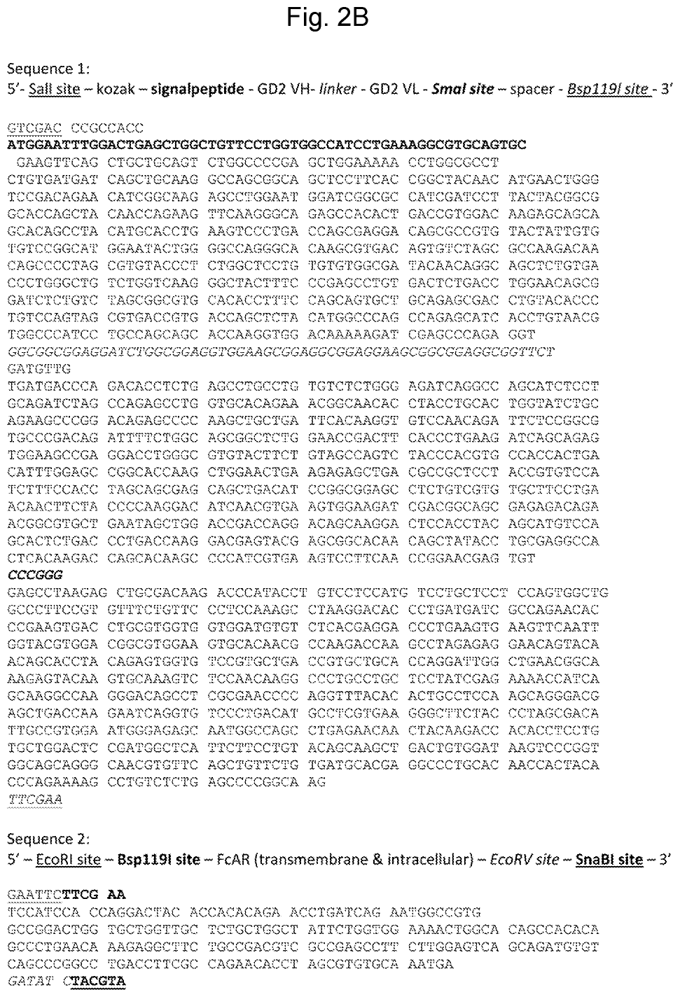

NB4, NK-92, neuroblastoma cell lines LAN-1, IMR-32, NMB, Ewing sarcoma cell line TC-71, and breast cancer cell line SKBR3 (ATCC) were cultured in IMDM medium (Thermo Fisher Scientific) supplemented with 20% (v/v) fetal bovine serum, penicillin (Sigma Aldrich, 100 U/mL), streptomycin (Sigma Aldrich, 100 μg/mL), and t-glutamine (Sigma Aldrich, 2 mM) and cultured at 37° C. in 5% CO2. Culture medium of NK-92 cells was supplemented with IL-2 (Peprotech, 100 units/mL). Epidermoid carcinoma cell line A431 (ATCC) was cultured in RPMI medium (Thermo Fisher Scientific) supplemented with 10% (v/v) fetal bovine serum, penicillin (Sigma Aldrich, 100 U/mL), streptomycin (Sigma Aldrich, 100 μg/mL), and t-glutamine (Sigma Aldrich, 2 mM) and cultured at 37° C. in 5% CO2. NB4 cells were differentiated towards neutrophil-like cells by All Trans Retinoic Acid (ATRA) (Sigma Aldrich, 0.5*106 cells/mL with 5 μmol ATRA/L) for 7 days. A431 cells overexpressing Her2/neu were generated by lentiviral transduction. Briefly, the Her2/neu coding sequence was ordered at Thermo Fisher Scientific and cloned into pENTR1A. Following recombination with lentiviral vector pRRL PPT SFFV prester SIN—Gateway B, the lentiviral construct pSin-Her2/neu was created, which was used for transduction of A431 cells. Cells expressing Her2/neu were selected by cell sorting. A431 cells were kept in culture in RPMI medium (Thermo Fisher Scientific) supplemented with 10% (v/v) fetal bovine serum, penicillin (Sigma Aldrich, 100 U/mL), streptomycin (Sigma Aldrich, 100 μg/mL), and t-glutamine (Sigma Aldrich, 2 mM) and cultured at 37° C. in 5% CO2. For GD2-CAR expression in WT NB4 cells, the coding sequences of the heavy and light chain variable (scFv) of the anti-GD2 antibody dinutuximab were connected via a linker and coupled to the intracellular tail of the intracellular part of the FcαR (see FIG. 2A for a schematic overview and FIG. 2B for details of the sequence). For Her2/neu-CAR expression in WT NB4 cells, the coding sequence of the scFv of the anti-Her2/neu antibody trastuzumab were connected to the intracellular tail of the intracellular part of the FcαR as described above for the GD2-CAR. For EGFR-CAR expression in NB4 cells, the coding sequence of the scFv of the anti-EGFR antibody cetuximab were connected to the intracellular tail of the intracellular part of the FcαR as described above for the GD2-CAR.

### CAR Constructs

Synthetic sequences were ordered at Thermo Fisher Scientific. Sequences were codon optimized for expression in human cells using the codon optimization service provided by the company website.

First, FcαR (transmembrane & intracellular) was cloned into the EcoRI-EcoRV sites of pENTR1A, and a Bsp119I restriction site was also introduced. Correct cloning was checked by restriction enzyme analysis on agarose gel. Next, the GD2-CAR-linker, Her2/neu-CAR-linker, or EGFR-CAR-linker fragment; each with CH2CH3 domains of human IgG1 (hinge), was cloned into the SalI-Bsp119I site of pENTR1A—FcαR (transmembrane & intracellular), and correct cloning was checked by restriction enzyme analysis on agarose gel. The construct pENTR1A—GD2-CAR-linker-hinge-FcαR (transmembrane & intracellular) construct was used to generate GD2-CAR fusions with either FcαR (transmembrane) or FcγRIIa (transmembrane & intracellular) by swapping the Bsp119I-EcoRV fragment. For generation of the IRES GFP construct, first the IRES GFP sequence (also containing a 5′ SnaBI restriction site) of LZRS mcs IRES GFP was cloned into the EcoRI-NotI sites of pENTR1A. Subsequent cloning of all CAR—IRES GFP constructs was similar as described for the constructs without IRES GFP, except that the SnaBI restriction site was used instead of the EcoRV restriction site. A V5 tag was introduced into pENTR1A—GD2-CAR-linker-hinge-FcαR (transmembrane & intracellular) IRES GFP by PCR amplification of the hinge, using these primers:
Forward: 5′ aatagctggaccgaccagg 3′, annealing in the VL region of GD2 CAR
Reverse: 5′ gagatc ttcgaa ggt gga gte gag gcc cag cag ag atta gg aat g ctt gcc accggt ctt gcc ggg gct cag aga cag 3′, with a Bsp119I restriction site in bold, an AgeI restriction site in italic, the V5 sequence (codon optimized for expression in human cells) underlined, and in bold and underlined the sequence annealing in the hinge.

A PCR using these primers on pENTR1A—GD2-CAR-linker-hinge-FcαR (transmembrane & intracellular) IRES GFP created a fragment containing part of GD2-CAR VL, internal SmaI restriction site, hinge, V5 tag and Bsp119I restriction site. This SmaI-Bsp119I fragment was cloned into the SmaI-Bsp119I sites of pENTR1A—GD2-CAR-linker-hinge-FcγRIIa (transmembrane & intracellular) IRES GFP, thereby replacing the original hinge for the hinge including the V5 tag. Correct cloning was checked by restriction enzyme analysis on agarose gel and sequencing.

Dual CAR constructs were generated by addition of a V5-tagged construct with IVS IRES Cherry. First, the IVS IRES sequence of pIRESPuro2 (Clontech) was PCR amplified using these primers:
Forward: 5′ gagatcgaattcgaattaattcgctgtctgcga 3′ with EcoRI restriction site in bold
Reverse: 5′ gagatcaccggtcatggaaggtcgtctccttg 3′ with AgeI restriction site in italic (IVS=synthetic intron, known to increase the stability of the mRNA, volgens de manual van Clontech)

After digestion and gel purification, the PCR product was cloned into the EcoRI-AgeI sites of pmCherry-N1 (Clontech). Correct cloning was checked by restriction enzyme analysis on agarose gel and sequencing. Next, an EcoRV restriction site was introduced into the EcoRI site of pmCherry-N1—IVS IRES, using oligo 5′ aattccggatatccgg 3′. This fragment, after annealing, will create a dsDNA fragment with EcoRI overhang. Correct cloning was checked by restriction enzyme analysis on agarose gel. Next, the EcoRV-NotI fragment containing IVS IRES Cherry was cloned into the SnaBI-NotI sites of pENTR1A—GD2-CAR-linker-hinge-V5-FcγRIIa (transmembrane & intracellular) IRES GFP, thereby replacing the IRES GFP for IVS IRES Cherry. Correct cloning was checked by restriction enzyme analysis on agarose gel.

The resulting constructs pENTR1A—GD2-CAR-FcαR (transmembrane & intracellular) All final pENTR1A constructs were subsequently recombined with pRRL PPT SFFV prester SIN—Gateway B.

For cloning digestions, approximately 1 μg of plasmid was digested with FastDigest enzyme (Thermo Fisher Scientific) in 1× Fast Digest buffer for over 20 minutes at 37° C. Digestions were run on 1% agarose; the correct fragments were sliced out with a clean surgical blade and purified using the QiaQuick Gel Extraction Kit (Biorad); as the final step the DNA was eluted in H₂O.

For ligation, vector and insert were incubated with Rapid T4 ligase in 1× Rapid Ligation Buffer (Thermo Fisher Scientific) for over 10 minutes at room temperature. For Gateway recombination, ˜150 ng of pRRL PPT SFFV prester SIN—Gateway B and ˜100 ng of the pENTR1A construct was added to Tris-EDTA (TE) buffer (pH 8.0), after which LR CLonase II (Thermo Fisher Scientific) was added, and after gently mixing the reaction was incubated overnight at 25° C. For transformation of E. coli DH5a, ligation product was added to DH5a, and incubated for 8 minutes on ice, followed by 45 seconds of heatshock at 42° C. The reaction was then incubated on ice for 2 minutes, after which Lysogeny broth (LB) was added and incubated for about 30-60 minutes at 37° C. and 200 rpm, after which the entire reaction was plated on LB-agar containing 30 μg/ml kanamycin (for pENTR1A constructs) or 100 μg/ml ampicillin (for pRRL PPT SFFV constructs), and the plates were incubated overnight at 37° C.

Single colonies were picked and used to inoculate LB+30 μg/ml kanamycin or 100 μg/ml ampicillin, and grown overnight at 37° C. and 200 rpm. The next day, overnight culture was used to isolate miniprep DNA using Nucleospin Plasmid EasyPure kit (Bioké). To check the minipreps, miniprep DNA was digested with FastDigest enzyme in 1× FastDigest buffer for over 20 minutes at 37° C., and digestions were run on a 1% agarose gel. Maxipreps were performed on overnight cultures using Nucleobond Xtra Maxi kit (Bioké).

To check the maxipreps, about 400 ng of maxiprep DNA was digested with FastDigest enzyme in 1× FastDigest buffer for over 20 minutes at 37° C., and digestions were run on a 1% agarose gel. Constructs were not additionally sequenced.

HEK293T cells were used to produce lentiviral particles, and were co-transfected with lentiviral vector, pMDLgp, pRSCrev and pCMV-VSVg in IMDM medium (with additives as described above). Two days after transfection, the lentivirus containing supernatant was filtered through a 0.45 μM filter and added to NB4 cells, after which the cells were passed on in lentiviral-free medium after two days. The cells were sorted on scFv anti-GD2 antibody expression by flow cytometry with the use of BiotinSP AffiniPure F(ab′)2 (Jackson Immunoresearch, 1 μg/mL) and Streptavidin Alexafluor 647 (Life technologies, 10 μg/mL), before their use in assays.

### Adhesion

NB4 cells (5*10⁶ cells/mL) were fluorescently labeled with Calcein-AM (Molecular Probes, 1 μmol/L) for 30 minutes at 37° C. and incubated in an uncoated 96-well MaxiSorp plate (Nunc, 2*106 cells/mL) in HEPES+(7.7 g NaCl (Fagron), 4,775 g HEPES (Sigma Aldrich), 450 mg KCl (Merck), 250 mg MgSO4 (Merck), 275 mg K2HPO4 (Merck), H2O (Gibco), pH 7.4 with 10 mol/L NaOH), with albumin (Albuman, Sanquin Plasma Products, 200 μg/mL), glucose (Merck, 1 mg/mL), and calcium (Calbiotech, cat. 208291, 1 mol/L). Cells were incubated for 30 minutes at 37° C. in 5% C02 in the presence of different stimuli: dithiothreitol (DTT) (Sigma Aldrich, 10 mmol/L), Pam3Cys (EMN Microcollections, 20 mg/mL), C5a (Sigma Aldrich, 10 nmol/L), tumor necrosis factor α (TNFα) (Peprotech, 10 ng/mL), phorbol 12-myristate 13-acetate (PMA) (Sigma Aldrich, 100 ng/mL), platelet-activation factor (PAF) (Sigma Aldrich, 100 nmol/L), N-formulmethionine-leucyl-phenylalanine (fMLP) (Sigma Aldrich, 30 nmol/L), or HEPES+ medium. The plate was washed twice with PBS and the cells were lysed at room temperature for 10 minutes with Triton (Sigma Aldrich, 0.5% X-100). A 100% lysed input of Calcein-labeled NB4 cells was used as control. Adhesion is determined in a Genios plate reader (Tecan) at an excitation wavelength of 485 nm and an emission wavelength of 535 nm.

### NADPH Oxidase Activity

ROS production was measured with an Amplex Red assay, determining the extracellular hydrogen peroxide release of NB4 cells after stimulation. NB4 cells (1*10⁶ cells/mL) were incubated for 5 minutes at 37° C. with Amplex Red (molecular probes, 20 mM) and horseradish peroxidase (Sigma Aldrich, 200 U/mL). Cells were activated with unopsonized zymosan (MP Biomedicals, 1 mg/mL), serum treated zymosan (STZ)37, PMA (Sigma Aldrich, 100 ng/mL), fMLP (Sigma Aldrich, 30 nmol/L), PAF (Sigma Aldrich, 100 nmol/L)/fMLP (Sigma Aldrich, 30 nmol/L), and HEPES⁺ as a negative control. The fluorescence was measured with a Genios plate reader (Tecan) for 30 minutes with 30 seconds intervals. The concentration H₂O₂ produced was determined from a calibration curve at an excitation wavelength of 535 nm and an emission wavelength of 595 nm with a 2 minute interval. Results are presented as the maximal slope in relative fluorescence units/minute.

### Western Blot

NB4 GD2-CAR were examined on western blot for expression of the scFv of the anti-GD2 antibody. 5*106NB4 cells were washed in PBS and resuspended in 50 μL Complete Protease Inhibitor Cocktail (Roche diagnostics)/ethylene diaminetetraacetic acid (EDTA) (0.45 mol/L) and 50 μL of 2× sample buffer (25 mL Tris B (Invitrogen), 20 mL 100% glycerol (Sigma Aldrich), 5 g sodium dodecyl sulphate (SDS) (Serva), 1.54 g DTT (Sigma Aldrich), 20 mg bromophenol blue (Sigma Aldrich), 1.7 mL 6-mercaptoethanol (Bio-Rad) and H2O to 50 mL (Gibco) at 95° C. for 30 minutes while vortexing every 10 minutes. For electrophoresis, 1*10⁶ cells were loaded into a 10% SDS-polyacrylamide gel electrophoresis (PAGE) gel and ran at 80 to 120 Volt. A nitrocellulose membrane (GE Healthcare Life Science) was used to transfer the proteins, at 0.33 ampere for 1 hour. The membrane was blocked and stained in 5% Bovine serum albumin (BSA) (Sigma)/Tris-Buffered Saline, 0.1% Tween 20 (TBST) for 1 hour at room temperature. BiotinSP Affinipure f(ab)2 (Jackson Immunoresearch, 0, 1 μg/mL, overnight at 4° C.) was used to detect the f(ab)2 region of the GD2-CAR and IRDYE 680 streptavidin (LI-COR, 0.4 μg/mL, 1 hour at room temperature) was used for Odyssey (LI-COR Biosciences) analysis.

### Flow Cytometry

The expression of the scFv of the anti-GD2 antibody was detected with primary antibody BiotinSP Affinipure f(ab)2 anti-mouse (Jackson Immunoresearch, 1 μg/mL, 30 minutes at 4° C.), and visualized with Streptavidin alexafluor 647 (Life technologies, 10 μg/mL, 30 minutes at 4° C.) on BD FACSCantoII. The expression of the scFv of the anti-Her2/neu and anti-EGFR antibody was detected with primary antibody BiotinSP Affinipure f(ab)2 anti-human (Jackson Immunoresearch, 1 μg/mL, 30 minutes at 4° C.), and visualized with secondary antibody Streptavidin alexafluor 647 (Life technologies, 10 μg/mL, 30 minutes at 4° C.) on BD FACSCantoII.

Expression of differentiation markers CD11b, FcγRIII (CD16), FcγRII (CD32), FcγRI (CD64) was tested with flow cytometry before every ADCC assay to ensure maturation of NB4 cells. In short, cells were incubated with fluorescently labeled antibodies against the abovementioned markers in PBS/0.5% human serum albumin (HSA) for 30 min on ice. After washing twice, cells were resuspended in 100 μL PBS/0.5% HSA and measured on BD FACSCantoTT.

### Cytotoxicity Assay

The neuroblastoma cell lines LAN-1, IMR-32, NMB, Ewing sarcoma cell line TC-71, breast cancer cell line SKBR3 and epidermoid carcinoma A431 were used as target cells. Target cell lines were harvested by trypsin (1%, in PBS) treatment after which 0.5*10⁶ cells were labeled with 50 μCi ⁵¹Cr (Perkin-Elmer, USA) at 37° C. for 90 minutes in 150 μL IMDM/RPMI medium (described above). For the indicated experiments LAN-1, IMR-32, NMB and TC-71 cell were opsonized with dinutuximab (Unituxin, ch14.18, United Therapeutics, 1 μg/mL) in IMDM medium (described above). SKBR3 cells were opsonized as indicated with trastuzumab (Herceptin, Roche, 1 μg/mL), A431 were opsonized as indicated with cetuximab (Erbitux, Merck, 1 μg/mL). Target cells (5*10³ cells/well) and effector cells (2.5*10⁵ cells/well) (1:50 target cell: effector cell (T:E) ratio, or other T:E ratios as indicated in the figure) were co-incubated in a 96-well U-bottom tissue culture plate in IMDM (described above) at 37° C. and 5% C02 for four hours. Cytotoxicity was normalized to a 100% ⁵¹Cr release by 0.1% triton (Sigma Aldrich, TX-100). 30 μL of supernatant was analyzed in a microbeta2 reader for radioactivity (PerkinElmer).

The percentage of cytotoxicity was determined as [(experimental value− spontaneous release)/(maximum release− spontaneous value)*100%]. Conditions were tested in duplicate or triplicate.

### Trogocytosis Assay

The trogocytosis of target cells by differentiated NB4 cells was quantified using flow cytometry and measured by the uptake of tumor cell membrane by the NB4 cells. Tumor cells were stained with 2 μM lipophilic membrane dye DiD (1,1-Dioctadecyl-3,3,3,3-tetramethylindodicarbocyanine, Invitrogen). After labeling, target cells were washed twice with PBS. Cells were co-incubated at a T:E ratio of 1:5 (i.e. 50.000:250.000 cells) in the absence or presence of 0.5 μg/mL dinutuximab, trastuzumab or cetuximab as indicated in a U-bottom 96-well plate (Greiner Bio-One) for 60 minutes at 37° C. and 5% C02 in IMDM complete medium. After incubation, cells were fixed with STOPbuffer (PBS containing 20 mM NaF, 0.5% PFA and 1% BSA) and analyzed using flow cytometer Canto II (BD Biosciences). The NB4 cell population was assessed for the mean fluorescence intensity (MFI) of membrane dye DiO.

### Data Analysis and Statistics

Flowjo software (Tree Star, Inc, Ashland, OR, USA) was used to analyze flow cytometry data and Graphpad Prism version 8 (Graphpad software) was used to visualize adhesion, Amplex Red, NB4 cell differentiation, flow cytometry, and ADCC results. Statistical analysis was performed using Prism. For adhesion, Amplex Red and expression of differentiation markers, two-way ANOVA-test was used followed by Sidak post-hoc test. For cytotoxicity assays one-way ANOVA-test followed by Sidak post-hoc test was used.

### Results

### GD2-FcαR-CAR is Expressed by Differentiated NB4 Cells

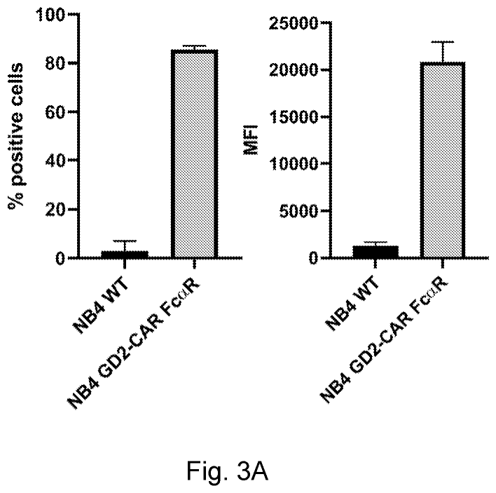

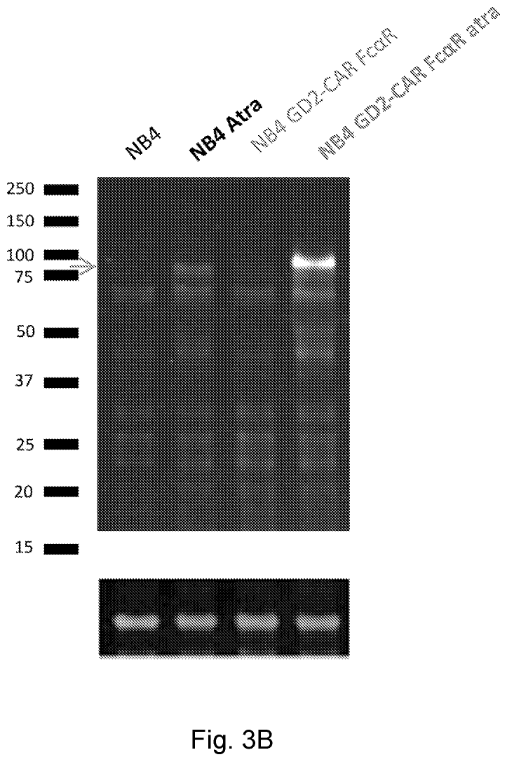

NB4 were differentiated towards neutrophil-like cells by 7 days stimulation with ATRA, after which the expression of the GD2-FcαR-CAR was evaluated by flow cytometry. FIG. 3A shows that more than 80% of the NB4 cells were positive for the GD2 FcαR-CAR. Also, NB4 cells that were transduced to express the GD2-FcαR-CAR showed high expression of the CAR as determined by MFI and compared to WT NB4 cells. Western blot (FIG. 3B) showed expression of the GD2-FcαR-CAR only in the ATRA differentiated NB4 cells transduced to express the CAR construct, at the expected height of ˜83 kDa.

### GD2-FcαR-CAR Expressing NB4 Cells are Able to Kill GD2+ Neuroblastoma Cell Lines

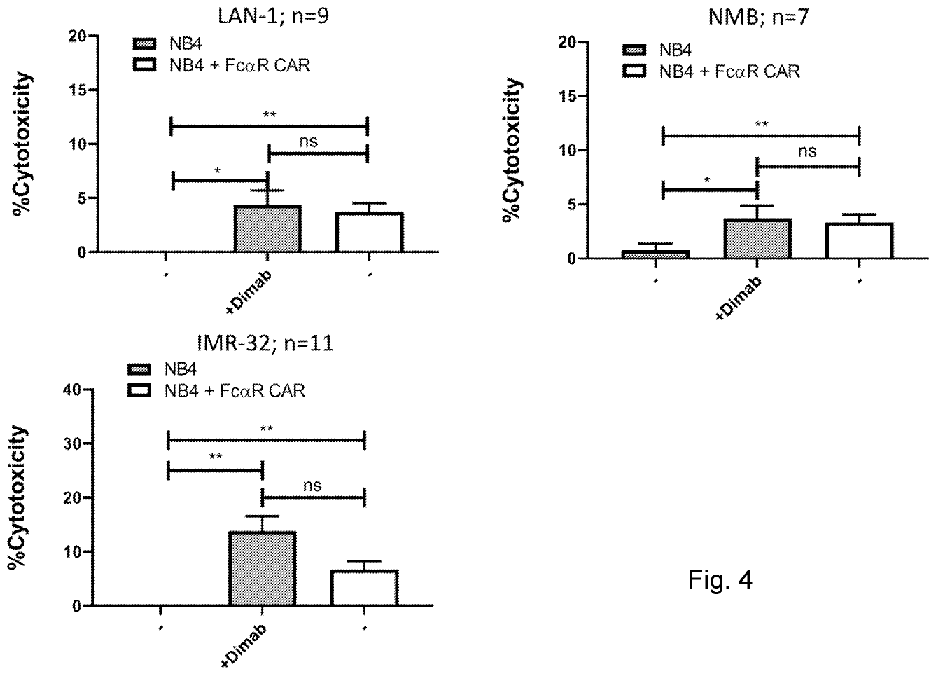

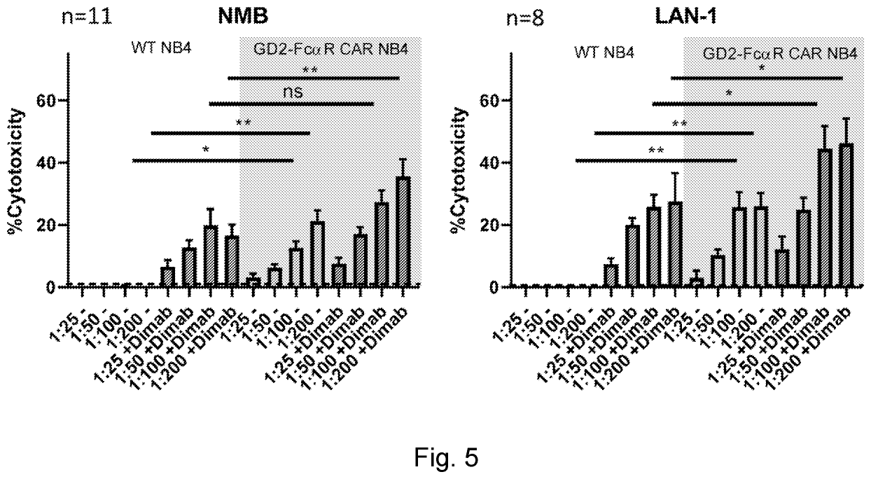

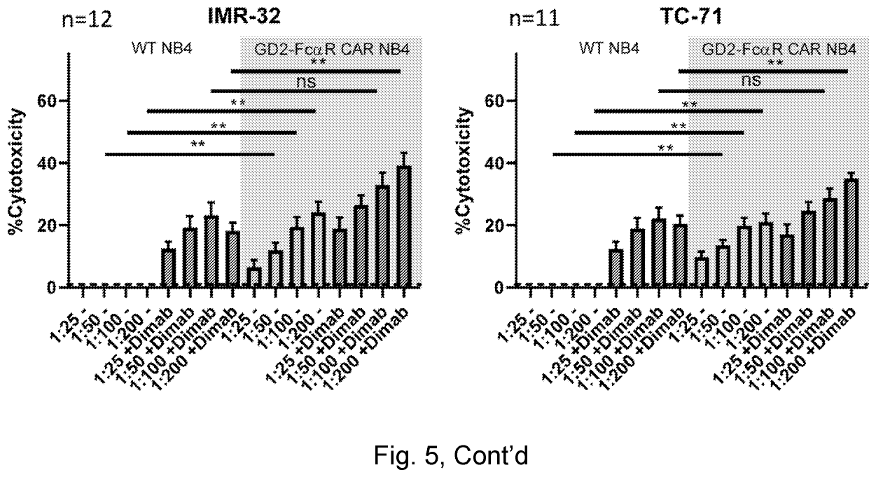

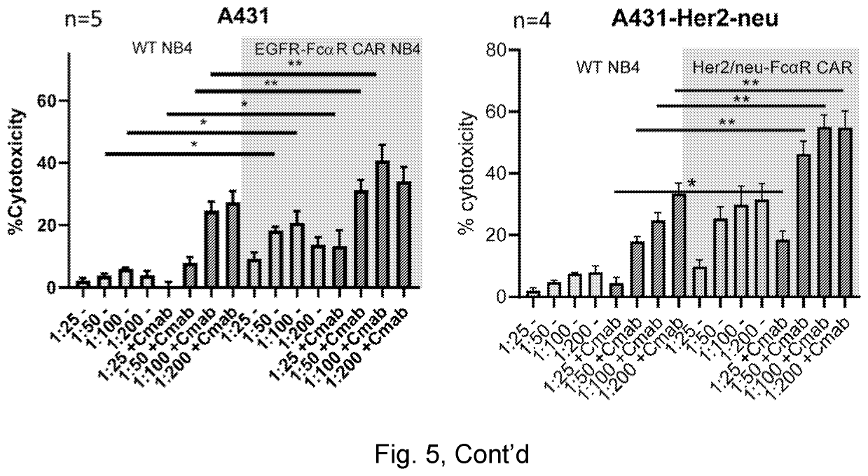

NB4 cells were differentiated towards neutrophil-like cells by 7 days ATRA stimulation and used in a cytotoxicity assay against GD2-positive neuroblastoma cell lines. As seen in FIG. 4, NB4 cells expressing the GD2-FcαR-CAR construct were capable of killing GD2+ neuroblastoma cell lines LAN-1, NMB and IMR-32, without the need for anti-GD2 opsonization. The cytotoxic capacity of the GD2 FcαR CAR against all neuroblastoma lines tested in non-opsonized condition was similar to the killing capacity of WT NB4 cells against dinutuximab-opsonized neuroblastoma cells. Non-opsonized neuroblastoma cells were not killed by WT NB4 cells. Titration of T:E ratios from 1:25 to 1:200 revealed a concentration dependent effect on cytotoxicity of the neuroblastoma cell lines LAN-1 and NMB. Again, GD2-FcαR-CAR expressing NB4 cells were able to kill the GD2+ neuroblastoma cell lines without the need for opsonisation with the ant-GD2 antibody dinutuximab. WT NB4 were not able to kill non-opsonized neuroblastoma cell lines in any of the T:E ratios. Opsonisation of the neuroblastoma cell lines with dinutuximab resulted in augmented cytotoxicity towards LAN-1 and NMB cells with increasing T:E ratios of the NB4 WT cells. This concentration dependent cytotoxicity towards the opsonized neuroblastoma cells was even more pronounced when the target cells were co-cultured in the presence of GD2-FcαR-CAR expressing NB4 cells (FIG. 5).

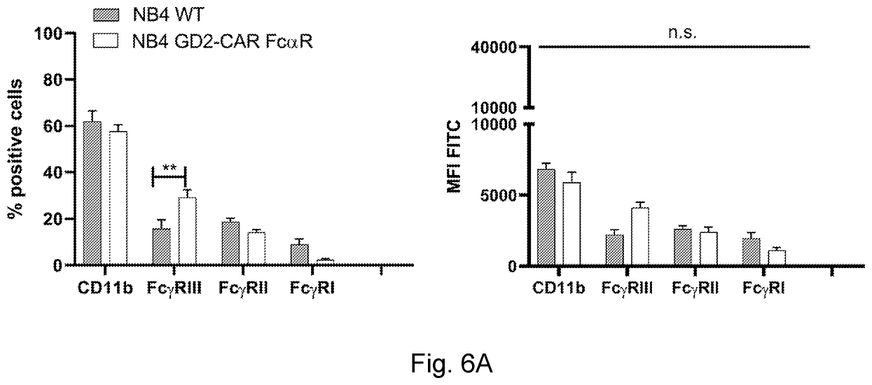

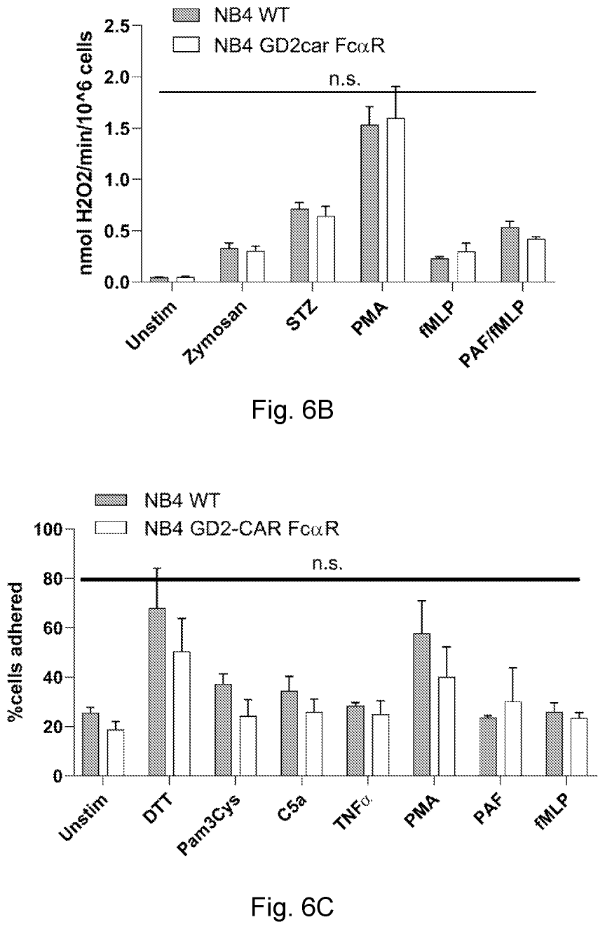

The differentiation of the NB4 cells, with and without expressing GD2-FCαR-CAR, was checked by flow cytometry after 7 days of ATRA differentiation. FIG. 6A shows the surface expression of several membrane markers, including CD11b and Fc-receptors, as a measure for sufficient differentiation of NB4 cells. Overall no difference in expression of the abovementioned surface markers was detected between WT NB4 cells and NB4 cells expressing the GD2-FcαR-CAR, indicating that the expression of the CAR does not hamper the differentiation process of these cells towards neutrophil-like cells. Furthermore, we evaluated two anti-microbial effector functions: the capability to produce ROS, and CD11b/CD18-mediated adhesion. As depicted in FIGS. 6B and C, both ROS production, as measured by Amplex Red assay, and adhesion did not significantly differ between WT NB4 cells and GD2-FcαR-CAR NB4 cells.

Together, our data indicate that GD2-FcαR-CAR expression does not hamper differentiation and effector functions of NB4 cells. Furthermore, the GD2-FcαR-CAR expressed on NB4 cells induce cytotoxicity of GD2-positive neuroblastoma cell lines, without the need for anti-GD2 opsonization.

Increased Cytotoxicity of FcαR CAR NB4 Cells Combined with Tumor Antigen Targeting Antibody Towards Several Different GD2+, EGFR+ and Her2/Neu+ Target Cells

As expansion of the dataset for NMB and LAN-1 cell lines as shown in FIG. 5, we increased the number of target cell lines. For the EGFR+ epidermoid carcinoma, and all GD2+ neuroblastoma and Ewing sarcoma cell lines tested the combination of EGFR/GD2-FcαR CAR in NB4 cells and anti-EGFR or anti-GD2 opsonizing antibody respectively on the tumor cells significantly increased the amount of cytotoxicity towards the tumor cells for the highest T:E ratio (1:200) (FIG. 5). For A431, the killing capacity of the EGFR-FcαR CAR increased after combination with the anti-EGFR antibody for all T:E ratios. In case of LAN-1, next to the 1:200 T:E ratio, also a T:E ratio of 1:100 significantly augmented cytotoxicity after the above-mentioned combination of GD2-FcαR CAR and anti-GD2-opsonizing antibody (see FIG. 5). A trend was observed towards increased killing for the lower T:E ratios (1:25, 1:50) in case of the GD2+ target cells, but these conditions did not reach significance under the circumstances tested. For the Her2/neu-targeting FcαR CAR the killing capacity of CAR-expressing NB4 cells could be further enhanced by combining with a tumor targeting antibody other than used in the CAR scFv antigen recognition part, in this case the anti-EGFR antibody cetuximab (see FIG. 5).

### GD2-FcαR-CAR is Antigen Specific

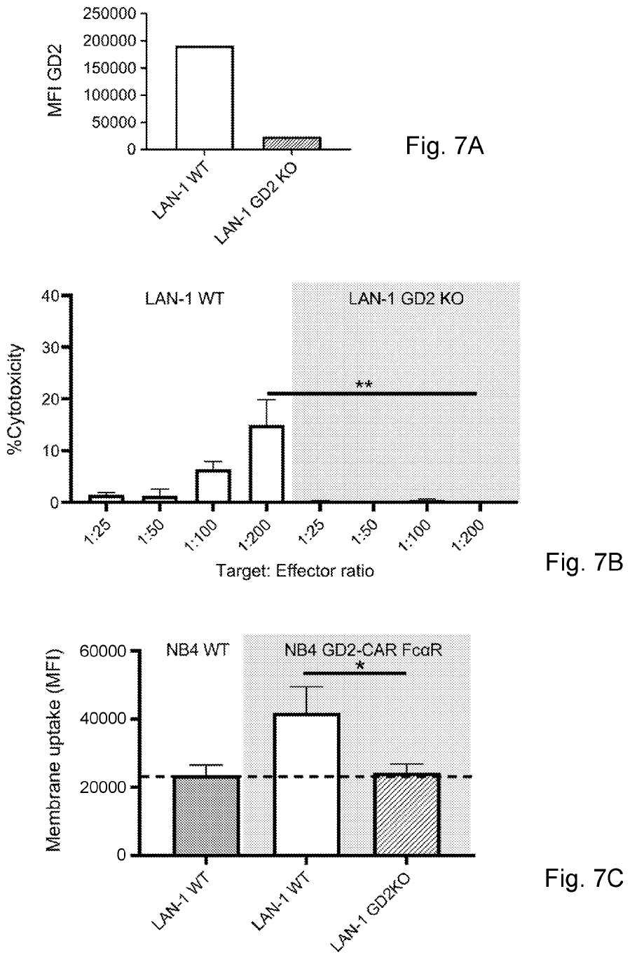

NB4 cells were differentiated towards neutrophil-like cells by 7 days ATRA stimulation and used in a cytotoxicity assay against GD2-positive and knockout neuroblastoma cell line LAN-1 (see FIG. 7A). FIG. 7B shows that NB4 cells expressing the GD2-FcαR-CAR construct were capable of killing GD2+ neuroblastoma cell lines LAN-1, but not GD2KO LAN-1 cells, indicating the GD2-FcαR-CAR is antigen specific. Trogocytosis, the recently described effector mechanism of neutrophils against antibody-opsonized tumor cells (Matlung et al., Cell Rep. 2018; 23(13):3946-3959.e6), showed a similar antigen specificity and overall pattern as was observed in the cytotoxicity assay using the GD2-FcαR-CAR (FIG. 7C).

### Functionality of GD2-FcαR CAR is Dependent on Expression of the FcαR Cytoplasmic Tail

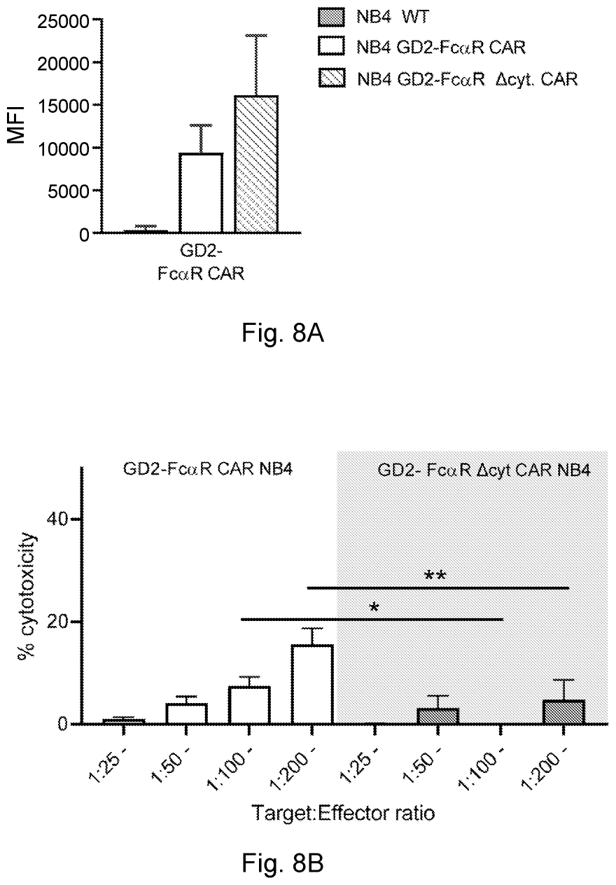

Next to the parental GD2-FcαR-CAR construct, we expressed the GD2-FcαR-CAR lacking the cytoplasmic domain of the FcαR (GD2-FcαR Δcyt CAR, FIG. 8A). These GD2-FcαR Δcyt CAR expressing NB4 cells were differentiated towards neutrophil-like cells by 7 days ATRA stimulation and used in a cytotoxicity assay against GD2-positive neuroblastoma cell line LAN-1. FIG. 8B shows that the cytotoxicity of these GD2-FcαR Δcyt CAR expressing NB4 cells was completely abrogated compared to the killing exerted by GD2-FcαR CAR NB4 cells, indicating that the FcαR cytoplasmic domain was indispensable for the function of the GD2-FcαR CAR.

### FcαR CAR is Effective Against Additional Tumor Antigens

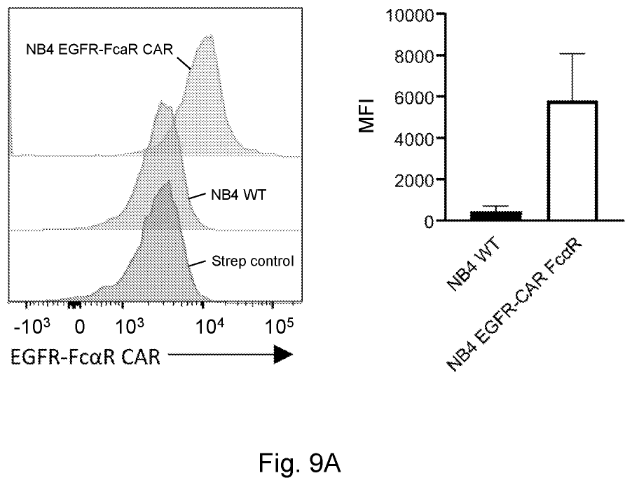

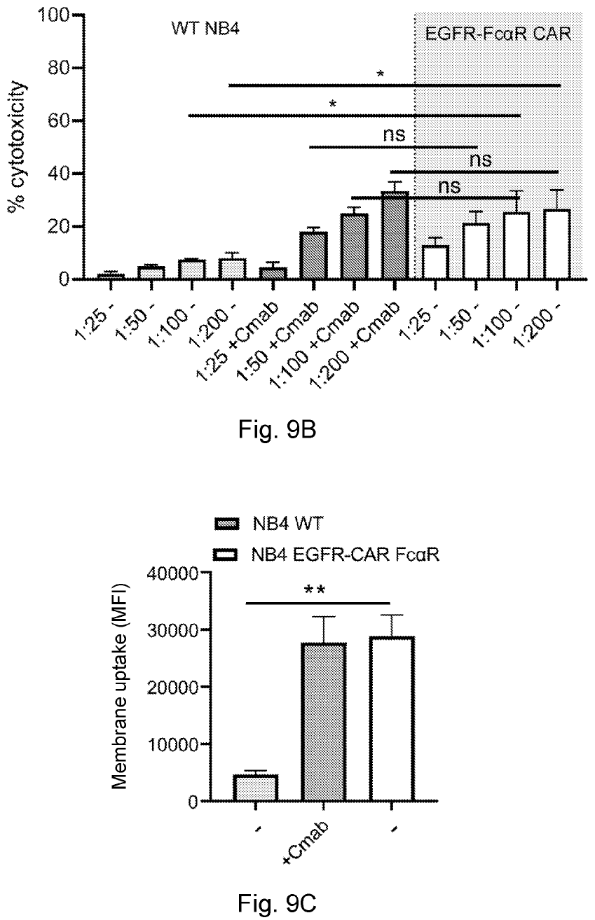

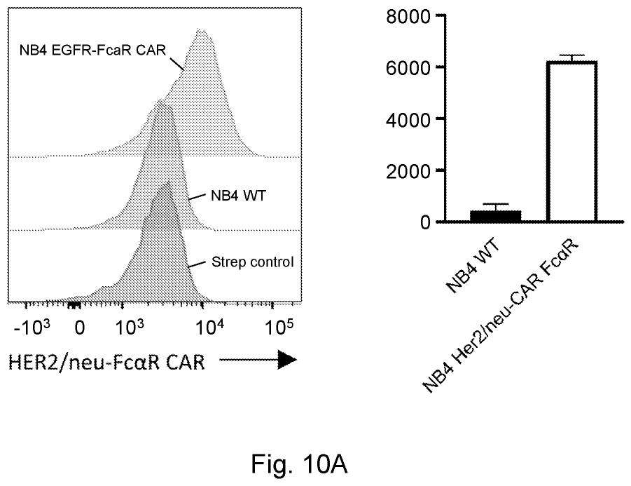

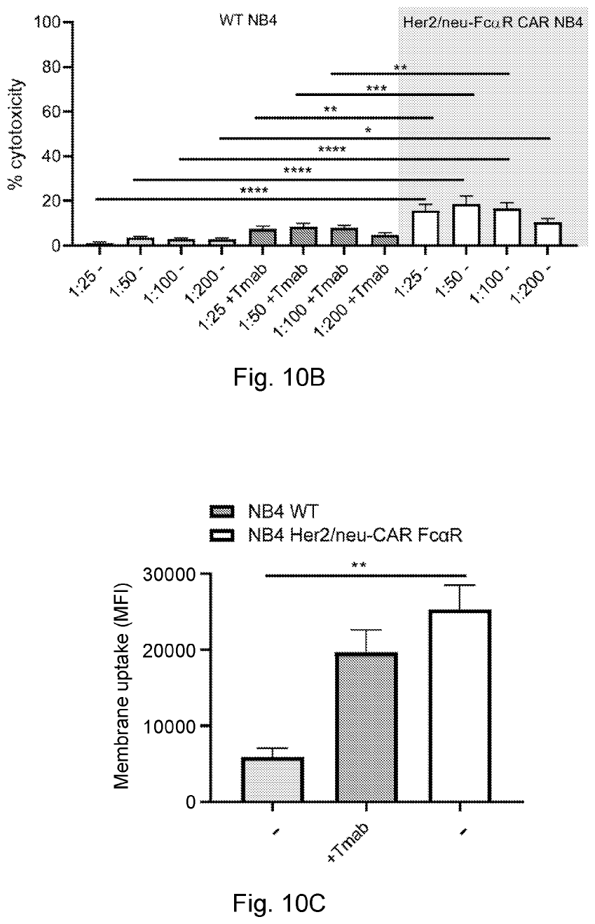

In addition to the GD2-targeting FcαR CAR, we generated FcαR CARs targeting tumor antigens EGFR (FIG. 9A) and Her2/neu (FIG. 10A) in NB4 cells. As seen in FIGS. 9B and 10B, NB4 cells expressing the EGFR and Her2/neu-FcαR-CAR constructs were capable of killing EGFR+ and Her2/neu+ epidermoid carcinoma and breast cancer cell lines A431 and SKBR3 respectively, without the need for anti-EGFR and anti-Her2/neu opsonization. The cytotoxic capacity of the EGFR-FcαR CAR in non-opsonized condition was similar to the killing capacity of WT NB4 cells against cetuximab-opsonized A431 cells (FIG. 9B) and even outperforms killing of the trastuzumab-opsonized SKBR3 cells (FIG. 10B). Non-opsonized target cells were not killed by WT NB4 cells at any of the T:E ratios. Trogocytosis showed a similar overall pattern as was observed in the cytotoxicity assay using the EGFR-FcαR-CAR (FIG. 9C) and Her2/neu-FcαR-CAR (FIG. 10C).

### GD2-FcαR CAR Outperforms GD2-FcγR CAR

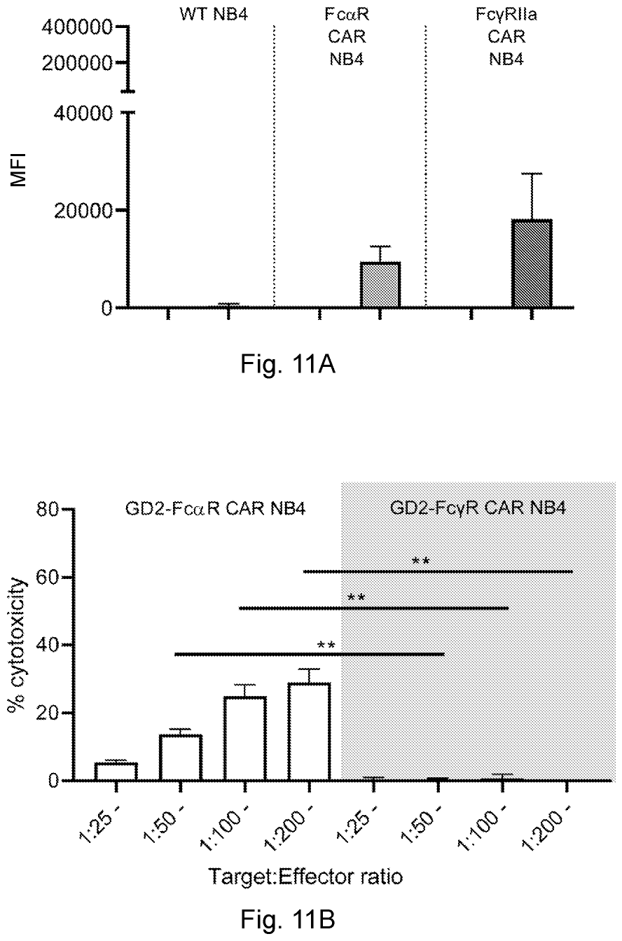

We generated two different CAR constructs in order to investigate their cytotoxic capacities: a GD2-targeting CAR construct comprised of the cytoplasmic domain of either FcαR or FcγRIIA. The expression of the two CAR constructs was similar if not higher for the GD2-FcγRIIA CAR, as detected by flow cytometry (FIG. 11A). As can be clearly seen from FIG. 11B, the cytotoxicity induced by the GD2-FcαR CAR in NB4 cells outperforms the GD2-FcγRIIA CAR in NB4 cells for all T:E ratios. More specifically, the expression of the GD2-FcγIIA CAR in NB4 cells did not induce killing of GD2+ target cells.

### Dual Expression of GD2-FcαR CAR and GD2-FcγR CAR Enhances Cytotoxicity Against GD2+ Tumor Cells

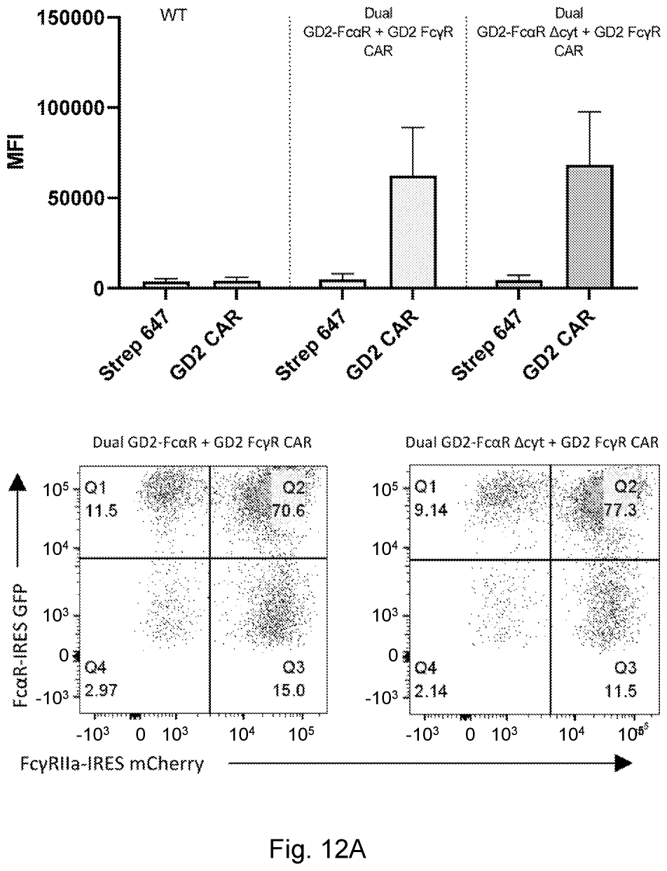

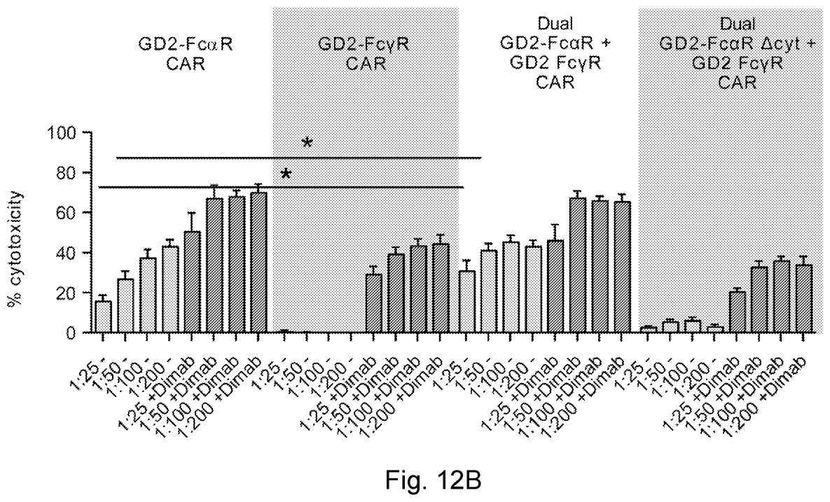

Furthermore, to investigate the possible cross-talk between FcαR and FcγR signalling we expressed multiple CARs in the same NB4 cell culture. FIG. 12A shows the successful dual expression of GD2-FcαR CAR and GD2-FcγRIIA CAR in NB4 cells. Moreover, we expressed the GD2-FcαR Δcyt CAR containing the truncated cytoplasmic domain of FcαR, in conjunction with the GD2-FcγRIIA CAR in NB4 cells (see FIG. 12A).

As shown previously, expression of the GD2-FcγRIIA CAR alone in NB4 cells did not induce antibody-independent killing of GD2⁺ LAN-1 target cells, whilst the GD2-FcγRIIA CAR expressing NB4 cells were capable of exerting their effector functions only when LAN-1 cells were opsonized with dinutuximab (FIG. 12B). Dual expression of the GD2-FcαR CAR and GD2-FcγRIIA CAR in NB4 cells enhanced the killing capacity towards LAN-1 cells; without the need for antibody opsonization compared to GD2-FcαR CAR alone. The further increase in killing capacity of these dual GD2-FcαR CAR and GD2-FcγRIIA CAR expressing NB4 cells after antibody-opsonization is most likely induced though simultaneous signalling by another Fc-receptor in addition to the FcαR CAR. The observed increase in antibody-dependent as well as antibody-independent killing furthermore is dependent on functional FcαR signalling, since expression of the truncated GD2-FcαR CAR reduced killing of LAN-1 cells back to baseline.

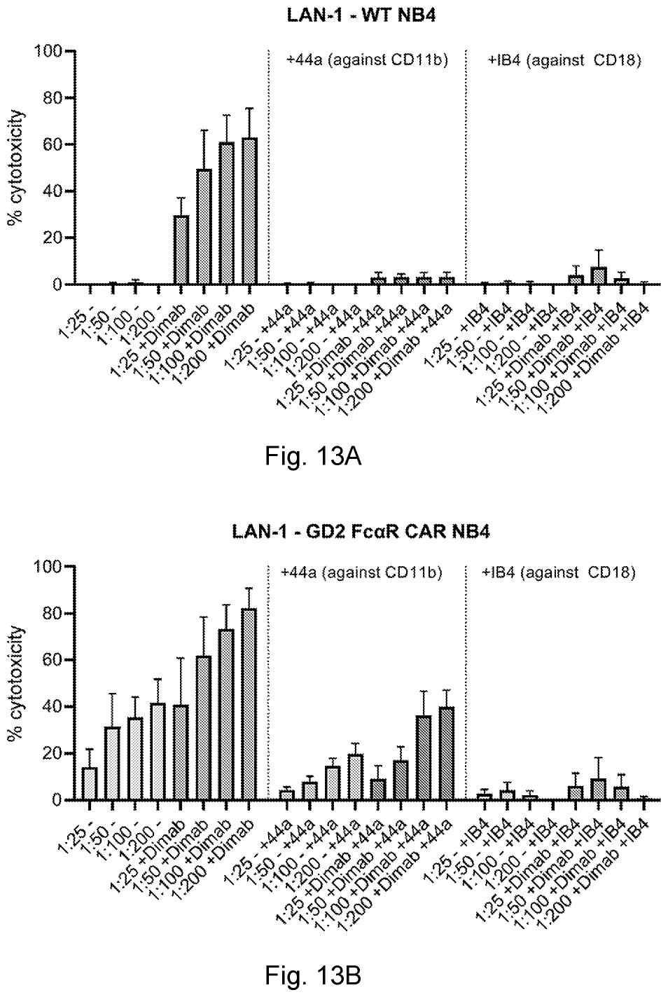

The mode of action behind the augmented killing observed for FcαR CAR in NB4 cells after combination with a tumor antigen-opsonizing antibody seems related to activation of additional 62-integrin(s) compared to antibody-induced killing after FcγR activation. FIGS. 13A and B show that blocking antibodies against either CD11b and CD18 can completely inhibit anti-GD2 and FcγR-induced killing, while this cytotoxicity is reduced but not completely inhibited after anti-CD11b treatment in case of GD2-FcαR CAR induced killing. Anti-CD18 treatment on the other hand is able to completely abrogate the antibody-dependent and -independent killing induced by the GD2-FcαR CAR (FIG. 13B).

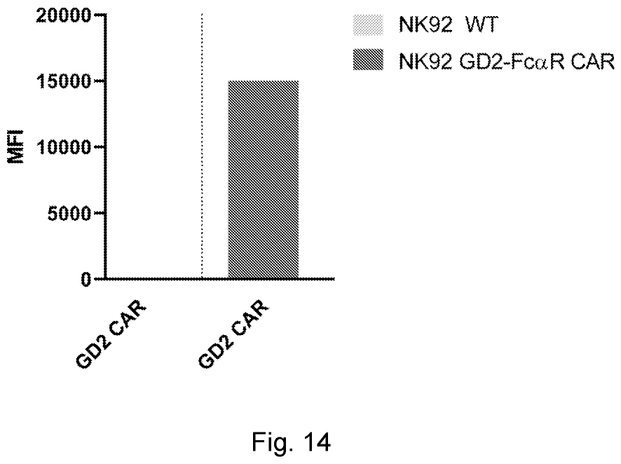

GD2-FcαR-CAR is expressed by NK-92 cells Finally, the expression of the GD2-FcαR-CAR in NK-92 cells was evaluated by flow cytometry. FIG. 14 shows that NK-92 cells that were transduced to express the GD2-FcαR-CAR showed high expression of the CAR as determined by MFI and compared to WT NK-92 cells (FIG. 14).

### REFERENCES

De Oliveira, S.; Ryan, C.; Giannoni, F.; Hardee, C. L.; Tremcinska, I.; Katebian, B.; Wherley, J.; Sahaghian, A.; Tu, A.; Grogan, T.; et al. Modification of Hematopoietic Stem/Progenitor Cells with CD19-Specific Chimeric Antigen Receptors as a Novel Approach for Cancer Immunotherapy. Hum. Gene Ther. 2013, 24, 824-839.
Guedan S, Calderon H, Posey A D Jr, Maus M V. Engineering and Design of Chimeric Antigen Receptors. Mol Ther Methods Clin Dev. 2018 Dec. 31; 12:145-156. doi: 10.1016/j.omtm.2018.12.009.
Johnson, A.; Song, Q.; Ferrigno, P. K.; Bueno, P. R.; Davis, J. Sensitive Affimer and Antibody Based Impedimetric Label-Free Assays for C-Reactive ProteinJ. Anal. Chem. 2012, 84, 6553-6560, DOI: 10.1021/ac300835b
Koristka S, Ziller-Walter P, Bergmann R, Arndt C, Feldmann A, Kegler A, Cartellieri M, Ehninger A, Ehninger G, Bornhauser M, Bachmann M P. Anti-CAR-engineered T cells for epitope-based elimination of autologous CAR T cells. Cancer Immunol Immunother. 2019 September; 68(9):1401-1415. doi: 10.1007/s00262-019-02376-y.
Mitwasi N, Feldmann A, Arndt C, Koristka S, Berndt N, Jureczek J, Loureiro L R, Bergmann R, Máthé D, Hegedüs N, Kovács T, Zhang C, Oberoi P, Jäger E, Seliger B, Rössig C, Temme A, Eitler J, Tonn T, Schmitz M, Hassel J C, Jäger D, Wels W S, Bachmann M. “UniCAR”-modified off-the-shelf NK-92 cells for targeting of GD2-expressing tumour cells. Sci Rep. 2020 Feb. 7; 10(1):2141. doi: 10.1038/s41598-020-59082-4.
Roberts, M. R.; Cooke, K. S.; Tran, A. C.; A Smith, K.; Lin, W. Y.; Wang, M.; Dull, T. J.; Farson, D.; Zsebo, K. M.; Finer, M. H. Antigen-specific cytolysis by neutrophils and NK cells expressing chimeric immune receptors bearing zeta or gamma signaling domains. J. Immunol. 1998, 161, 375-384.
Sievers N M, Dorrie J, Schaft N. CARs: Beyond T Cells and T Cell-Derived Signaling Domains. Int J Mol Sci. 2020 May 15; 21(10):3525. doi: 10.3390/ijms21103525.
Schäfer D, Henze J, Pfeifer R, Schleicher A, Brauner J, Mockel-Tenbrinck N, Barth C, Gudert D, Al Rawashdeh W, Johnston I C D, Hardt O. A Novel Siglec-4 Derived Spacer Improves the Functionality of CAR T Cells Against Membrane-Proximal Epitopes. Front Immunol. 2020 Aug. 7; 11:1704. doi: 10.3389/fimmu.2020.01704.
Tang X, Yang L, Li Z, Nalin A P, Dai H, Xu T, Yin J, You F, Zhu M, Shen W, Chen G, Zhu X, Wu D, Yu J. First-in-man clinical trial of CAR NK-92 cells: safety test of CD33-CAR NK-92 cells in patients with relapsed and refractory acute myeloid leukemia. Am J Cancer Res. 2018 Jun. 1; 8(6):1083-1089.

1-25. (canceled)

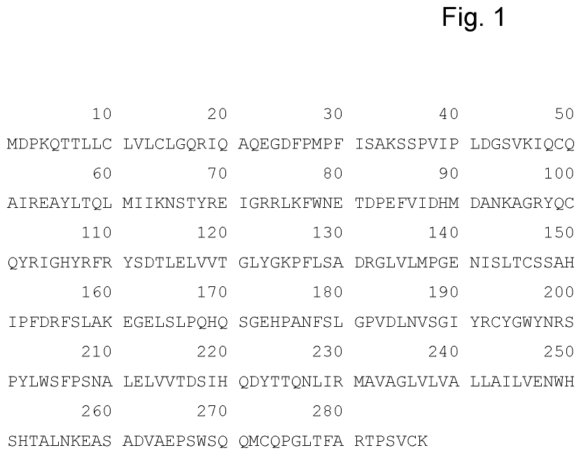

26. A neutrophil or NK cell expressing a chimeric antigen receptor (CAR) comprising a polypeptide comprising:
an intracellular domain of a Fc alpha Receptor (FcαR),
a transmembrane domain of a FcαR, and
a heterologous ligand-binding domain
wherein the polypeptide comprises amino acids 228 to 287 of the amino acid sequence of FcαR as depicted in FIG. 1 or a sequence that is at least 90% identical to said sequence of FcαR.

27. The neutrophil or NK cell according to claim 26 wherein the polypeptide further comprises a spacer located between the transmembrane domain of a FcαR and the ligand-binding domain.

28. The neutrophil or NK cell according to claim 26, wherein the ligand-binding domain is a domain specific for a cell surface antigen, such as a domain specific for a tumor antigen, or a myeloid derived suppressor cell antigen.

29. The neutrophil or NK cell according to claim 26, wherein the ligand-binding domain comprises an antibody or antigen binding part thereof, a nanobody or antigen binding fragment thereof, or an affimer.

30. The neutrophil or NK cell according to claim 26, wherein the ligand-binding domain is specific for GD2, EGFR or HER2/Neu.

31. The neutrophil or NK cell according to claim 26, wherein the polypeptide comprises amino acids 228 to 287 of the amino acid sequence of FcαR as depicted in FIG. 1.

32. A neutrophil or NK cell according to claim 26 wherein a nucleic acid molecule encoding the CAR or a vector comprising the nucleic acid molecule is introduced.

33. The neutrophil or NK cell according to claim 32 wherein the nucleic acid molecule or vector is introduced into said neutrophil or NK cell by transfection, transduction or electroporation.

34. The neutrophil or NK cell according to claim 26, wherein said neutrophil or NK cell is an autologous cell isolated from a patient suffering from cancer.

35. A population of cells comprising a plurality of neutrophils or NK cells according to claim 26.

36. A pharmaceutical composition comprising the neutrophil or NK cell according to claim 26 and at least one pharmaceutically acceptable carrier, diluent or excipient.

37. A method for immunotherapy in a subject in need thereof comprising administering to the subject thereof a therapeutically effective amount of the neutrophil or NK cell according to claim 26.

38. The method according to claim 37 for inducing or stimulating an immune response in a subject in need thereof comprising administering to the subject therapeutically effective amount of the neutrophil or NK cell.

39. The method according to claim 37 for the treatment of cancer in a subject in need thereof, comprising administering to the subject a therapeutically effective amount of the neutrophil or NK cell.

40. The method according to claim 37 for the treatment or prevention of a pathogenic infection in a subject in need thereof comprising administering to the subject a therapeutically effective amount of the neutrophil or NK cell.

41. The method according to claim 39 wherein said cancer is a solid tumor.

42. The method according to claim 41, wherein the cancer is selected from the group consisting of neuroblastoma, melanoma, small cell lung cancer (SCLC), Ewing sarcoma, osteosarcoma, glioma, retinoblastoma, breast cancer, bladder cancer, colon cancer, head and neck cancer, non-small cell lung cancer (NSCLC), anal cancers, and glioblastoma.

43. A method of producing a population of the neutrophils or NK cells according to claim 26, comprising:
introducing a nucleic acid molecule encoding the chimeric antigen receptor (CAR) in cells of a population of neutrophils or NK cells, and
allowing expression of the CAR.

44. The neutrophil or NK cell according to claim 26, wherein the polypeptide comprises amino acids 228 to 287 of the amino acid sequence of FcαR as depicted in FIG. 1 or the corresponding sequence of a FcαR isoform other than isoform A.1.

45. The method according to claim 26, wherein said neutrophil or population of cells is combined with a therapeutic antibody, a checkpoint inhibitor, cytokine, chemotherapeutic agent, or T cell based therapy, preferably with a therapeutic antibody.
